(19) United States
(12) Patent Application Publication
(10) Pub. No.: US 2012/0172336 A2
(43) Pub. Date: Jul. 5, 2012

(54) Fosfluconazole Derivatives, Synthesis, and Use in Long Acting Formulations

(75) Inventors: Yves Gonnissen, Tongeren (BE); Jody Voorspoels, Reningelst (BE)

(73) Assignee: SEPS Pharma N.V., Tongeren (BE)

(21) Appl. No.: 13/256,875

(22) PCT Filed: Mar. 3, 2010
(86) PCT No.: PCT/EP2010/052719

§ 371 Date: Sep. 15, 2011

(30) Foreign Application Priority Data
Mar. 19, 2009 (GB) 0904706.9
Oct. 27, 2009 (EP) 09174129.8

(51) Int. Cl. A61K 31/675; A61K 31/683; C07J 43/00; A61P 31/10; C07F 9/146; C07F 9/24; C07F 9/08; C07F 9/6518; A61K 31/685

(52) U.S. Cl. 514/93; 548/112; 540/5; 558/202; 558/199

(57) ABSTRACT

The invention relates to a compound of formula (1) and the salts, N-oxides, quaternary amines, and stereoisomers thereof, wherein R¹ to R² are as defined in the claims. The invention further relates to intermediates and methods for the preparation of the compounds of formula (I). The invention also relates to the compounds of formula (I) for use as a medicament, particularly for the prevention or treatment of fungal infections.

### FIELD OF THE INVENTION

Fuconazole, also known as Diflucan ®, is a triazole antifungal agent firstly described in UK patent application 2099818 (Pfizer Limited). It is used worldwide for the treatment of infections due to Candida, Cryptococcus, and other opportunistic yeasts or fungi. The drug is available as a tablet (50, 100, or 200 mg), as an oral suspension, and as an intravenous formulation (200 or 400 mg). When used in the treatment of invasive candidiasis, e.g., bloodstream infections, deep tissue sites, or other normally sterile site infections, fluconazole is administered as an initial loading dose of 800 mg (oral or intravenous) followed by a daily maintenance dose of 400 mg (oral or intravenous). Higher daily doses of 800 mg or greater may be used in selected Circumstances (J. Infect. 1993, 16:133-146. Clin Infect. Dis. 2004, 38:161-189. Clin. Infect. Dis. 2003, 36:1221-1228. Eur. J. Clin. Microbiol. Infect. Dis. 1999, 18:165-174).

Phosphates are extensively used to prepare water soluble prodrugs for intravenous administration. Patent application WO9728169 (Pfizer) discloses phosphate esters of fluconazole, particularly fosfluconazole or Prodif®, which prodrug results from the esterification of the hydroxyl group of fluconazole with phosphoric acid. In the body, it rapidly hydrolyzes thereby exhibiting clinical effects equivalent to those of fluconazole. Phosphate esterification of fluconazole has endowed the compound with high solubility in aqueous solution of pH 4 to 12. A volume of 200 mL used to be necessary for administering 400 mg fluconazole, whereas as little as 5 mL of solution is needed to administer 400 mg fluconazole-equivalent of fosfluconazole, a 40-fold reduction in the volume, thereby permitting bolus injection. In patients with deep-seated mycosis requiring high dose antifungal agents, multiple concomitant medication as well as adjuvant therapy such as fluid replacement is performed. However, in patients complicated by serious underlying diseases, particularly cardiac failure, respiratory failure, or ascites, fluid replacement may be restricted to adjust the balance of water content and electrolytes in the body. Compared with fluconazole, fosfluconazole is easier to use in patients with deep-seated mycosis because it can be administered by bolus injection resulting in a marked decrease in volume load. (Japanese Pharmacology & Therapeutics 2005, 33(4), 267-302).

Daily maintenance doses of fluconazole are a serious constraint to the effective treatment of fungal infections. Such dosage schedules lead to a higher workload for clinical personnel and more importantly, poor patient compliance, thereby increasing the probability of administering suboptimal doses, ultimately contributing to the emergence of resistant fungal strains. It has been established that reduced access of the drug to the target enzyme, the fungal cytochrome P-450-dependent enzyme lanosterol 14-α-demethylase, is one of the mechanisms that produce resistance in Candida albicans (Clin. Microbiol. Rev. 1999, 12:501-517). Also, exposure of C. glabrata to subtherapeutic doses (i.e., <400 mg/day) of fluconazole may result in resistance not only to fluconazole but to other azoles (i.e., itraconazole and voriconazole) as well (Antimicrob, Agent Chemother. 2005, 49:783-787). It is also interesting to note that overexpression of the target enzyme encoding gene ERG11 results in the production of high concentrations of the target enzyme, creating the need for higher intracellular fluconazole concentrations to inhibit all of the enzyme molecules in the cell.

When patient compliance is a problem, long acting dosage forms of medication is a possible solution, where a single administration leads to a sustained release of the medication over an extended period of time. Such dosage forms simplify the regimen that a patient needs to adhere to, thereby reducing the probability of non-compliance as occurs with a more rigorous schedule. Among such dosage forms is the depot formulation, which can be administered in various ways including intramuscularly by injection. The depot dosage injection is formulated to provide slow absorption of the drug from the site of administration, often keeping therapeutic levels in the patients system for days or weeks at a time.

Depot dosage injections do not come free of charge. The higher the volume to be injected, the more painful the injection is. Jorgensen et al. (Ann Pharmacother. 1996; 30(7-8): 729-32) have shown a correlation between pain and the volume of a subcutaneous injection with volumes of 1-1.5 mL causing significantly more pain than volumes of 0.5 mL or less. It is therefore desirable to minimize injection volume wherever possible.

Patent application WO05006860 (The Board of Governors for Higher Education State of Rhode Island and Providence Plantations) discloses phosphate triesters linked to fatty alcohols.

Nguyen-Hai Nam et al. (Bioorg Med Chem. 2004 Dec 1; 12(23):6255-69) synthesized and evaluated fatty alcohol and carbohydrate phosphate esters of fluconazole.

It is an object of the present invention to provide fluconazole derivatives and formulations that deliver the drug over a sustained period of time at concentrations efficacious for treatment of mammals including humans. Such fluconazole forms and formulations must be safe, i.e. having minimal side effects, and with appropriate pharmacokinetic profiles.

It is an object of the present invention to provide fluconazole derivatives and formulations that can improve one or more of the following pharmacokinetic parameters in respect of the currently available formulations: a longer half-life, increased volume of distribution, extended drug release, sustained plasma concentration, or a longer duration of action.

It is an object of the present invention to provide derivatives and formulations of fluconazole in High loading doses when compared to the currently available formulations.

It is an object of the present invention to provide chemically stable derivatives of fluconazole. It is a further object of the present invention to provide chemically stable formulations of fluconazole. It is also an object of the present invention to provide soluble derivatives of fluconazole

It is an object of the present invention to minimize the number of doses of fluconazole to be administered. It is an object of the present invention to provide fluconazole derivatives or formulations that allow bolus injection. It is another object of the present invention to provide fluconazole depots in a patient.

It is an object of the present invention to provide fluconazole injectable formulations with a suitable volume that avoids painful administration.

It is an object of the present invention to provide derivatives and formulations of fluconazole that can reduce the emergence of resistance fungal strains.

It is an object of the present invention to provide derivatives and formulations of fluconazole that can increase intracellular fluconazole concentrations.

### SUMMARY OF THE INVENTION

The inventors have surprisingly found certain fosfluconazole derivatives with interesting solubility profiles, exhibiting valuable high solubilities in certain lipophilic solvents. In particular, certain fosfluconazole derivatives of the present invention are highly suitable for lipidic formulations and injectable depot formulations. These formulations exhibit effective plasma concentrations even two months after administration. In addition, the invention achieves this ultimately desired increased drug loading and prolonged delivery in a convenient mode of administration both for the patient and clinical personnel.

The present invention relates to a compound of formula (I) 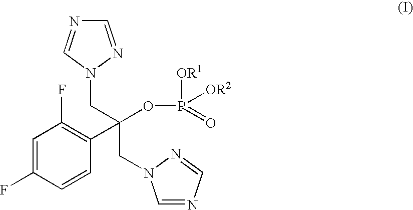
and the salts, N-oxides, quaternary amines, and stereoisomers thereof, wherein R¹ is hydrogen or C₁₋₆alkyl;
R² is a steranyl or a group selected from 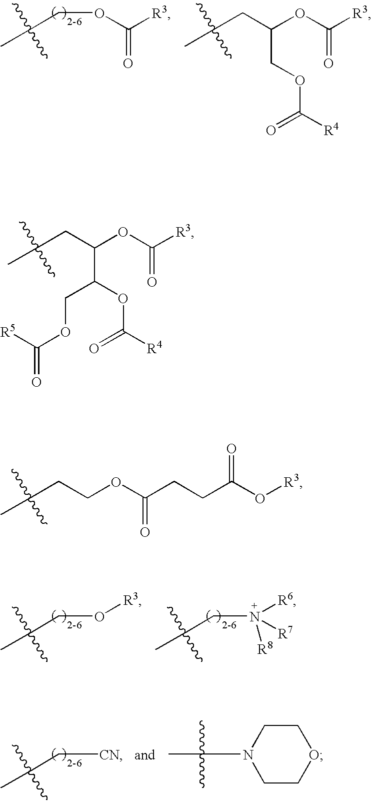
the symbol 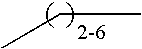
   represents a C₂₋₆alkanediyl;
R³, R⁴, and R⁵ are each independently, C₆₋₁₈alkyl or steranyl; and
R⁶, R⁷, and R⁸ are each, independently, C₁₋₆alkyl.

The invention further relates to methods for the preparation of the compounds of formula (I), the salts and stereochemically isomeric forms thereof, and to intermediates used in these preparation methods.

The invention also relates to the compounds of formula (I) per se, the salts, N-oxides, quaternary amines, and stereochemically isomeric forms thereof, for use as a medicament. The invention also relates to pharmaceutical compositions comprising a pharmaceutically acceptable carrier and an effective amount of a compound of formula (I) as specified herein.

The invention further relates to the aforementioned compounds, compositions, and pharmaceutical compositions for the manufacture of a medicament for the prevention or treatment of fungal infections. Or expressed in other words, the invention relates to compounds, compositions, and pharmaceutical compositions for use in the prevention or treatment of fungal infections. Similarly, the invention relates to a method for the prevention or treatment of fungal infections by administering an effective amount of the compounds, compositions, and pharmaceutical compositions as described herein to a patient in need thereof.

The invention also relates to a method of improving the lipophilicity of fluconazole, or extending the release or pharmacological activity of fluconazole, which methods comprise the conversion of fluconazole into a compound of formula (I), or a pharmaceutically acceptable salt thereof.

The invention further relates to the use of the chemical group of formula (VI) as a promoiety 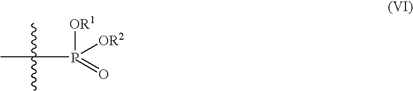
wherein R¹ and R² are each as defined above; and
the wavy line (depicted by [symbol] ) indicates the bond to the oxygen atom of a drug.

### DESCRIPTION OF THE FIGURES

FIG. 1: Melting point characterization measured by Differential Scanning Calorimetry (DSC) of 2-(2,4-difluorophenyl)-1,3-di(1H-1,2,4-triazol-1-yl)propan-2-yl ethyl (3R,8R,9S,10R,13S,14S,17R)-10,13,17-trimethyl-17-((R)-6-methylheptan-2-yl)-2,3,4,7,8,9,10,11,12,13,14,15,16,17-tetradecahydro-1H-cyclopenta[a]phenanthren-3-yl phosphate

FIG. 2: Melting point DSC characterization of 2-((2-(2,4-difluorophenyl)-1,3-di(1H-1,2,4-triazol-1-yl)propan-2-yloxy)(ethoxy)phosphoryloxy)ethyl(3S,8R,9S,10R,13S,14S,17R)-10,13,17-trimethyl-17-((R)-6-methylheptan-2-yl)-2,3,4,7,8,9,10,11,12,13,14,15,16,17 -tetradecahydro-1H-cyclopenta[a]phenanthren-3-yl succinate

### DESCRIPTION OF THE INVENTION

The present invention relates to a compound of formula (I) 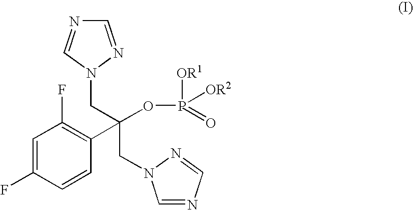
and the salts, N-oxides, quaternary amines, and stereoisomers thereof, wherein
R¹ is hydrogen or C₁₋₆alkyl'
R² is a steranyl or a group selected from 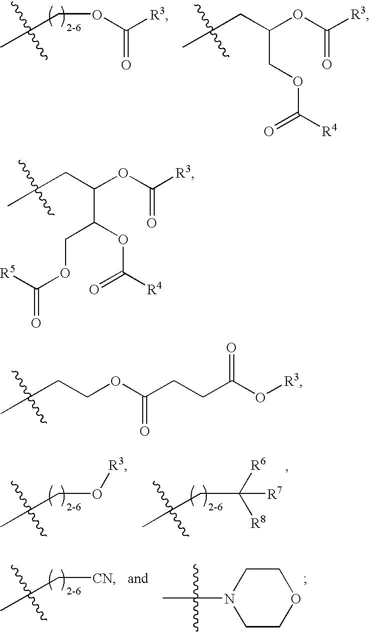
the symbol 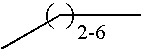
   represents a C₂₋₆alkanediyl;
R³, R⁴, and R⁵ are each independently, C₆₋₁₈alkyl or steranyl; and
R⁶, R⁷, and R⁸ are each, independently, C₁₋₆alkyl.

As used in the foregoing and hereinafter, the following definitions apply, unless otherwise noted.

As used herein “C₁₋₄alkyl” as a group or part of a group defines straight or branched chain saturated hydrocarbon radicals having from 1 to 4 carbon atoms such as for example methyl, ethyl, 1-propyl, 2-propyl, 1-butyl, 2-butyl, 2-methyl-1-propyl.

“C₁₋₆alkyl” encompasses C₁₋₄alkyl radicals and the higher homologues thereof having 5 or 6 carbon atoms such as, for example, 1-pentyl, 2-pentyl, 3-pentyl, 1-hexyl, 2-hexyl, 2-methyl-1-butyl, 2-methyl-1-pentyl, 2-ethyl-1-butyl, 3-methyl-2-pentyl, and the like. Of interest among C₁₋₆alkyl is C₁₋₄alkyl.

“C₆₋₁₈alkyl” as a group or part of a group defines straight or branched chain saturated Hydrocarbon radicals having from 6 to 18 carbon atoms such as for example heptanyl, octanyl, nonanyl, decanyl, undecanyl, dodecanyl, tridecanyl, tetradecanyl, pentadecanyl, hexadecanyl, heptadecanyl, octadecanyl, 4,8-dimethylhexadecanyl, 4-ethyl-11-methylpentadecanyl, 5-butyldodecanyl, and the like.

C₃₋₇cycloalkyl is generic to cyclopropyl, cyclobutyl, cyclopentyl, cyclohexyl, and cycloheptyl.

The term “C₂₋₆alkanediyl” is defined identically the same as the corresponding “C₂₋₆alkyl” but is bivalent instead of monovalent. As such, a bivalent C₂₋₆alkyl defines straight of branched chain saturated bivalent hydrocarbon radicals having from 2 to 6 carbon atoms such as 1,2-ethanediyl or 1,2-ethylene, 1,3-propanediyl or 1,3-propylene, 1,2-propanediyl or 1,2-propylene, 1,4-butanediyl or 1,4-butylene, 1,3-butanediyl or 1,3-butylene, 1,2-butanediyl or 1,2-butylene, 1,5-pentanediyl or 1,5-pentylene, 1,6-hexanediyl or 1,6-hexylene, ect., also including the alkylidene radicals such as ethylidene, propylidene, and the like.

The term “steranyl” refers both to steroid and sterol radicals that bind to the oxygen or carbon atoms of compound of formula (I), as the case may be, through that specific carbon atom bearing a hydroxyl group in the 4-cyclic sterane structure, said hydroxyl group being not present at the end compound of formula (I).

The term “steroid” refers to polycyclic compounds having a common nucleus, a fused, reduced 17-carbon atom ring system, cyclopentanoperhydrophenanthrene of formula (VII).

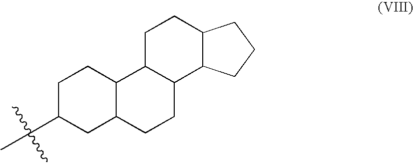

In one embodiment, the steroid radical has two methyl groups and an aliphatic side-chain attached to the nucleus. (From Hawley's Condensed Chemical Dictionary, 11th ed). In one embodiment, the steroid radical is the group of the formula (VIII), wherein the wavy line indicates the bond of attachment within the compound of formula (I)/

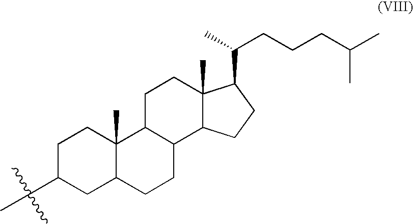

The term “sterol” refers to steroids with a hydroxyl group at C-3 and most of the skeleton of cholestane. Additional carbon atoms may be present in the side chain. (IUPAC Steroid Nomenclature, 1987).

In particular, the term “steranyl” encompasses the radical forms of adosterols; cholecalciferols such as hydroxycholecalciferol (calcifediol, dihydroxycholecalciferol, 24,25-dihydroxyvitamin D3, calcitriol); cholesterols such as cholesterol per se, 19-iodocholesterol, azacosterol, cholestanol, cholesterol esters, dehydrocholesterols (desmosterol), hydroxycholesterols, ketocholesterols, dihydrotachysterol; ergocalciferols such as 25-hydroxyvitamin D2; fusidic acid; lanosterol; phytosterols such as ecdysteroids, ergosterol (whitanolides), sitosterol, stigmasterol; cycloartenol; zoosterol; and derivates thereof. As an example of the terminology used in the present invention, the radical form of cholesterol is referred to herein as cholesteranyl. The radical form of cholestanol is referred to herein as cholestanyl.

In table 1 below, the chemical structures of interesting sterols and steroids are depicted.

TABLE 1
adosterol

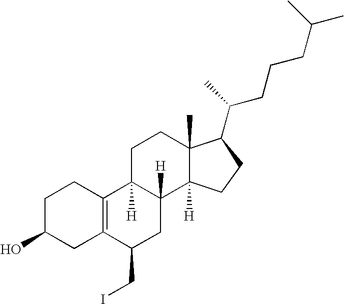

azacosterol

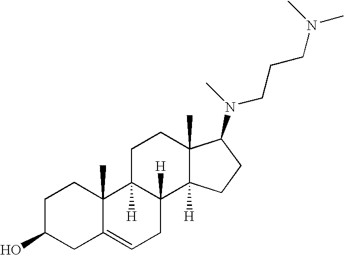

calcifediol

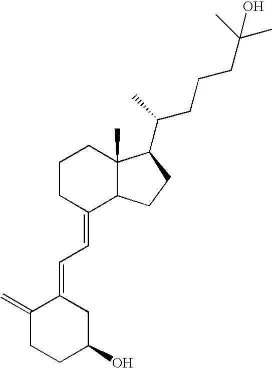

calcitriol

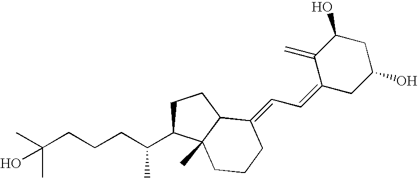

campesterol

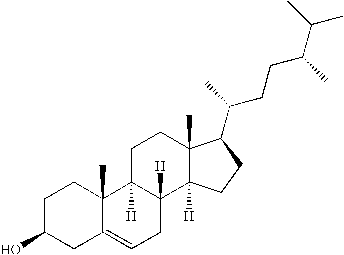

cholecalciferol

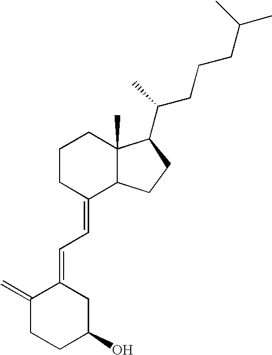

cholestanol

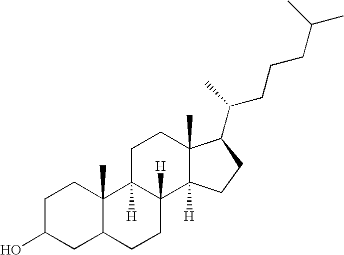

cholesterol

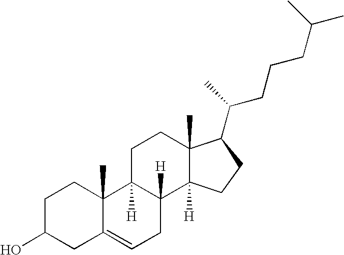

cycloartenol

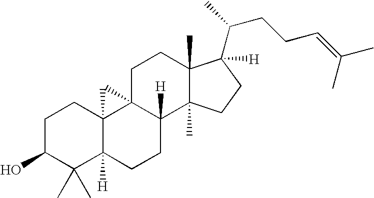

desmosterol

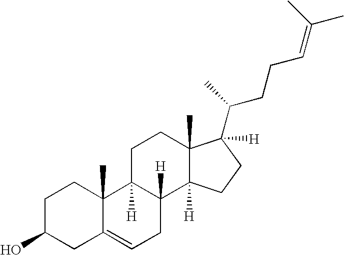

dihydrotachysterol

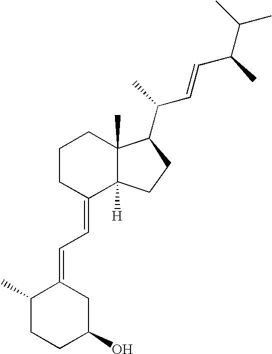

dihydroxycholecalciferol

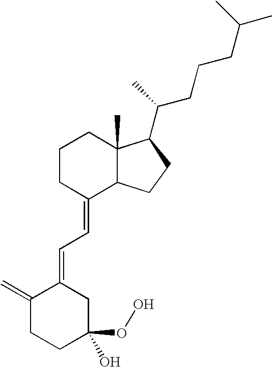

24,25-dihydroxyvitamin D3

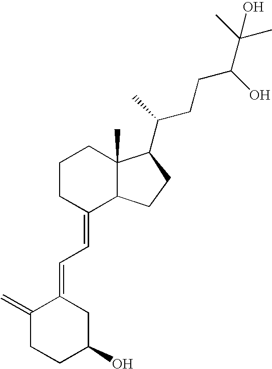

ergocalciferol

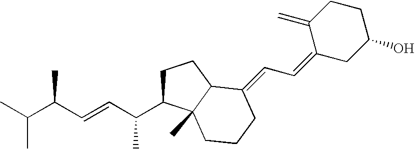

ergosterol

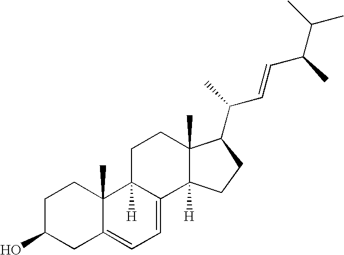

fusidic acid

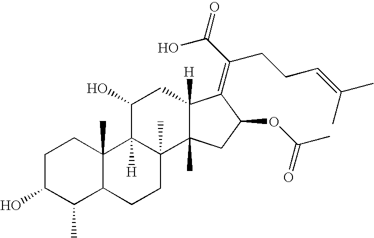

hydroxycholecalciferol

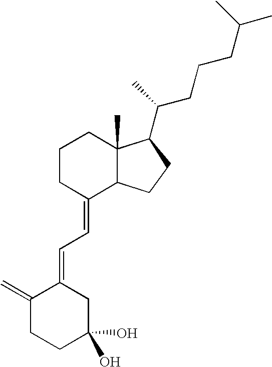

hydroxycholesterol

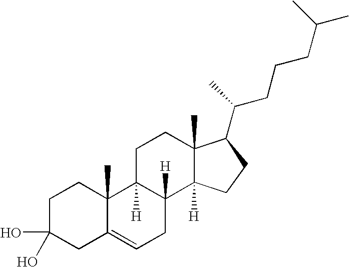

25-hydroxyvitamin D2

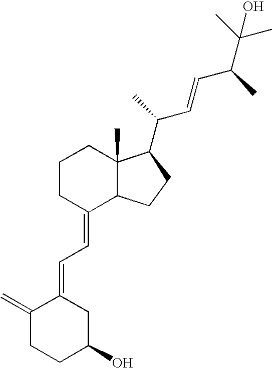

19-iodocholesterol

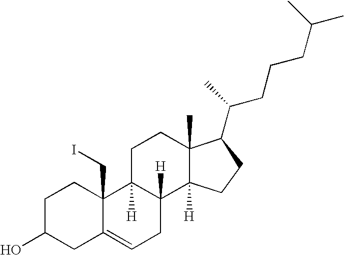

ketocholesterol

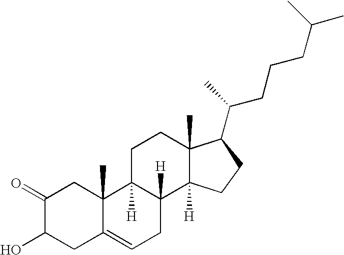

lanosterol

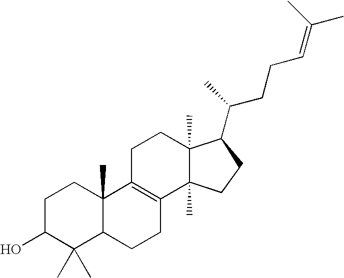

sitosterol

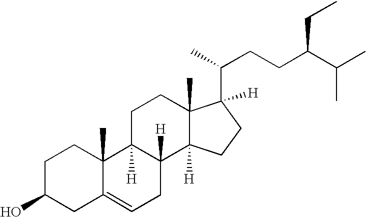

stigmasterol

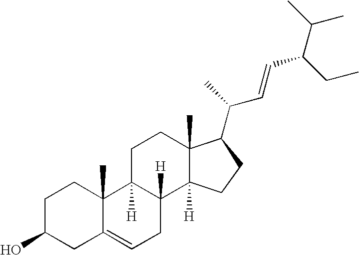

It should be noted that the radical positions on any molecular moiety used in the definitions may be anywhere on such moiety as long as it is chemically stable. When any variable occurs more than one time in any moiety, each definition is independent. Radicals used in the definitions of the variables include all possible isomers unless otherwise indicated. For instance pentyl includes 1-pentyl, 2-pentyl and 3-pentyl.

Whenever used hereinafter, the term “compounds of formula (I)”, or “the present compounds” or similar terms, it is meant to include the compounds of formula (I), the salts thereof; and the stereochemically isomeric forms thereof.

The compounds of formula (I) may have several centers of chirality, particularly when R² or R³ is a steranyl, and exist as stereochemically isomeric forms. The term “stereochemically isomeric forms” as used herein defines all the possible compounds made up of the same atoms bonded by the same sequence of bonds but having different three-dimensional structures which are not interchangeable, which the compounds of formula (I) may possess. With reference to the instances where (R) or (S) is used to designate the absolute configuration of a chiral atom within a substituent, the designation is done taking into consideration the whole compound and not the substituent in isolation.

Unless otherwise mentioned or indicated, the chemical designation of a compound encompasses the mixture of all possible stereochemically isomeric forms, which said compound might possess. Said mixture may contain all diastereomers and enantiomers of the basic molecular structure of said compound. All stereochemically isomeric forms of the compounds of the present invention both in pure form of mixed with each other are intended to be embraced within the scope of the present invention.

Pure stereoisomeric forms of the compounds and intermediates as mentioned herein are defined as isomers substantially free of other enantiomeric or diastercomeric forms of the same basic molecular structure of said compounds or intermediates. In particular, the term “stereoisomerically pure” concerns compounds or intermediates having a stereoisomeric excess of at least 80% (i.e. minimum 80% of one isomer and maximum 20% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms “enantiomerically pure” and “diastereomerically pure” should be understood in a similar way, but then having regard to the enantiomeric excess, and the diastereomeric excess, respectively, of the mixture in question.

Pure stereoisomeric forms of the compounds and intermediates of this invention may be obtained by the application of art-known procedures. For instance, enantiomers may be separated from each other by the selective crystallization of their diastereomeric salts with optically active acids or bases. Examples thereof are tartaric acid, dibenzoyltartaric acid, ditoluoyltartaric acid and camphorsulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occur stereospecifically. Preferably, if a specific stereoisomer is desired, said compound will be synthesized by stereospecific methods of preparation. These methods will advantageously employ enantiomerically pure starting materials.

The diastereomeric racemates of the compounds of formula (I) can be obtained separately by conventional methods. Appropriate physical separation methods that may advantageously be employed are, for example, selective crystallization and chromatography, e.g. column chromatography.

The present invention is also intended to include all isotopes of atoms occurring on the present compounds. Isotopes include those atoms having the same atomic number but different mass numbers. By was of general example and without limitation, isotopes of hydrogen include tritium and deuterium. Isotopes of carbon include C-13 and C-14.

For therapeutic use, salts of the compounds of formula (I) are those wherein the counter-ion is pharmaceutically acceptable, which salts can be referred to as pharmaceutically acceptable acid and base addition salts. However, salts of acids and bases that are non-pharmaceutically acceptable may also find use, for example, in the preparation or purification of a pharmaceutically acceptable compound. All salts, whether pharmaceutically acceptable or not, are included within the ambit of the present invention.

The pharmaceutically acceptable acid and base addition salts as mentioned hereinabove are meant to comprise the therapeutically active non-toxic acid and base addition salt forms that the compounds of formula (I) are able to form. The pharmaceutically acceptable acid addition salts can conveniently be obtained by treating the base form with such appropriate acid in an anion form. Appropriate anions comprise, for example, acetate, benzenesulfonate, benzoate, bicarbonate, bitartrate, bromide, calcium edetate, camsyiate, carbonate, chloride, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, (embonate), pantothenate, phosphate/diphosphate, polygalacturonate, salicylate, stearate, subacetate, succinate, sulfate, tannate, tartrate, teoclate, triethiodide, and the like. Conversly said salt forms can be converted by treatment with an appropriate base into the free base form.

The compounds of formula (I) containing an acidic proton may also be converted into their non-toxic metal or amine addition salt forms by treatment with appropriate organic and inorganic bases in a cation form. Appropriate basic salts comprise those formed with organic cations such as benzathine, chloroprocaine, choline, diethanolamine, ethylenediamine, meglumine, procaine, and the like; and those formed with metallic cations such as aluminum, calcium, lithium, magnesium, potassium, sodium, zinc, and the like. Conversely said salt forms can be converted by treatment with an appropriate acid into the free form.

The “N-oxide” forms of the present compounds are meant to comprise those wherein one or several nitrogen atoms in any one of the triazole rings are oxidized (e.g., mono-or di-oxide). The nitrogen mono-oxides may exist as a single positional isomer or a mixture of positional isomers (e.g., a mixture of 1-N-oxide, 2-N-oxide, and 4-N-oxide triazoles).

The term “quaternary amine” as used hereinbefore defines the quaternary ammonium salts which the compounds of formula (I) are able to form by reaction between a basic nitrogen of a compound of formula (I) and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins. Quaternary amines of compounds of formula (I) may be obtained by alkylating a nitrogen-containing heterocycle, i.e. one or the two triazole rings, with bromoethyl acetate to give a quaternary ammonium mono- or disalt.

Some of the compounds of formula (I) may also exist in their tautomeric form. Such forms, although not explicitly indicated in the above formula, are intended to be included within the scope of the present invention.

One embodiment of the present invention concerns compounds of formula (I) wherein one or more of the following conditions apply:

a) R¹ is hydrogen or C₁₋₆alkyl;

b) R² is a steranyl, or a group selected from

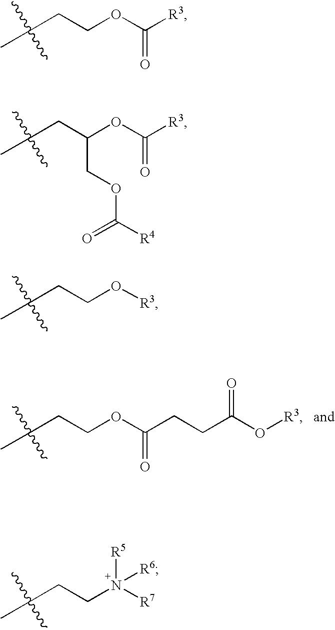

c) R³, R⁴, and R⁵ are each, independently, C₆₋₁₈alkyl or steranyl;

d) R⁶, R⁷, and R⁸ are each, independently, C₁₋₆alkyl.

One embodiment of the present invention concerns compounds of formula (I) wherein one or more of the following conditions apply:

a) R¹ is C₁₋₆alkyl;

b) R² is a steranyl or 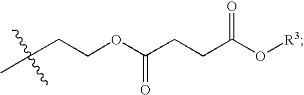
in which group, R³ is steranyl.

One embodiment of the present invention concerns compounds of formula (I) and any subgroup thereof wherein the steranyl is cholesteranyl.

One embodiment of the present invention concerns compounds of formula (I) and any subgroup thereof wherein R¹ is ethyl.

One embodiment of the present invention relates to a salt of the compound of formula (I) and any subgroup thereof, wherein R¹ is hydrogen and the salt is a monosodium salt.

One embodiment of the present invention relates to any one of the following compounds:
2-(2,4-difluorophenyl)-1,3-di(1H-1,2,4-triazol-1-yl)propan-2-yl 2-(trimethylammonio)-ethyl phosphate
sodium 2-(decanoyloxy)ethyl 2-(2,4-difluorophenyl)-1,3-di(1H-1,2,4-triazol-1-yl)propan-2-yl phosphate
sodium 2-(2,4-difluorophenyl)-1,3-di(1H-1,2,4-triazol-1-yl)propan-2-yl 2-(nonyloxy)ethyl phosphate
sodium 2,3-bis(decanoyloxy)propyl 2-(2,4-difluorophenyl)-1,3-di(1H-1,2,4-triazol-1-yl)propan-2-yl phosphate
2-(2,4-difluorophenyl)-1,3-di(1H-1,2,4-triazol-1-yl)propan-2-yl ethyl (3R,3R,9S,10R,13S,14S,17R)-10,13,17-trimethyl-17-((R)-6-methylheptan-2-yl)-2,3,4,7,8,9,10,11,12,13,14,15,16,17-tetradecahydro-1H-cyclopenta[a]phenanthren-3-yl phosphate
2-((2-(2,4-difluorophenyl)-1,3-di(1H-1,2,4-triazol-1-yl)propan-2-yloxy)(ethoxy)phosphoryloxy)ethyl (3S,8R,9S,10R,13S,14S,17R)-10,13,17-trimethyl-17 -((R)-6-methylheptan-2-yl)-2,3,4,7,8,9,10,11,12,13,14,15,16,17-tetradecahydro-1H-cyclopenta[a]phenanthren-3-yl succinate
sodium 2-(2,4-difluorophenyl)-1,3-di(1H-1,2,4-triazol-1-yl)propan-2-yl nonyl phosphate

The present invention further provides a process for the preparation of a compound of formula (I) as defined above, or a pharmaceutically acceptable salt thereof, which comprises phosphorylating fluconazole and when desired or necessary converting the resulting compound into a pharmaceutically acceptable salt or vice versa.

The phosphorylation may be carried out in different ways, some of them provided in patent publication WO97/28169 (Pfizer Limited). One method of phosphorylation (a1) may be accomplished by reacting fluconazole with a phosphoramidite of formula (II) in a suitable reaction medium thereby obtaining a phosphite of formula (III), and further reacting said phosphite of formula (III) with an oxidant,

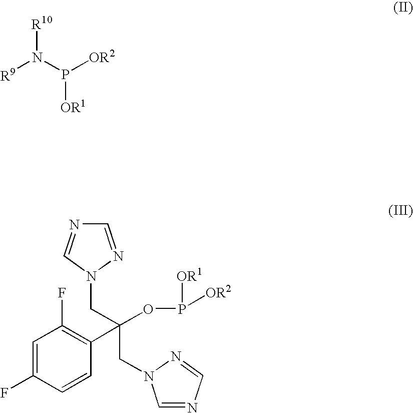

wherein

R¹ and R² are each as defined above;

R⁹ and R¹⁰ are each, independently, C₁₋₆alkyl optionally substituted with C₃₋₇cycloalkyl or phenyl, optionally substituted phenyl, or C₃₋₇cycloalkyl; or R⁹ and R¹⁰ together with the nitrogen atom to which they are attached form an optionally substituted 5- or 6-membered saturated heterocyclic ring, wherein the substituents may be selected from C₁₋₄alkyl and phenyl. Preferably, R⁹ and R¹⁰ are each, independently, C₁₋₆alkyl, phenyl, or together with the nitrogen atom to which they are attached form a morpholine ring.

The reaction may be carried out in a solvent which does not adversely influence the reaction, e.g. methylene chloride or tetrahydrofuran, in the presence of a mild acid such as tetrazole, 5-methyl-1H-tetrazole, 1,2,4-1H-triazole, pyridinium hydrobromide, or imidazole hydrochloride with, and optional catalytic 4-(dimethylamino)pyridine, at room temperature or above.

The resulting phosphite of formula (III) is then reacted with an oxidant, for example a peracid such as 3-chloroperoxybenzoic acid or H₂O₂, preferably a 30% aqueous hydrogen peroxide, to provide the final phosphate of formula (I). The reaction may be carried out in a solvent which does not adversely influence the reaction, e.g. methylene chloride or tetrahydrofuran, below room temperature, for example at 0 to 20° C.

An alternative method of phosphorylation (a2) is accomplished by reacting fluconazole with a phosphorochloridate of formula (IV) in a suitable anhydrous reaction medium such as acetone, dichloromethane, and potassium carbonate. The reaction may be carried out at room temperature.

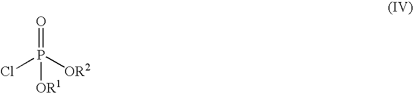

An additional method of phosphorylation (a3) may be achieved by reacting fluconazole with PCl₃ in the presence of a base thereby obtaining an intermediate compound of formula (V),
R—O—PCl₂ (V)
and reacting compound of formula (V) with a compound of formula R¹—OH and a compound of formula R²—OH;

wherein

R¹ and R² are each as defined above; and

R is

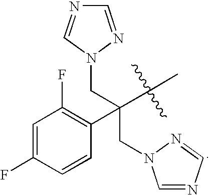

The reaction of fluconazole and PCl₃ may be carried out in a solvent which does not adversely affect the reaction, e.g. methylene chloride or tetrahydrofuran, at a temperature in the range −20 to +20° C., for example at 0° C. Suitable bases include pyridine and N-methylimidazole.

The sequential reactions of the compound of formula (V) with a compound of formula R¹—OH and with a compound of formula R²—OH (in which R¹ and R² are as defined above) to result into a compound of formula (I), as defined above, may be performed without isolation of the compound of formula (V), at a temperature around room temperature. These sequential reactions may be accomplished in any given order, e.g. firstly introducing compound of formula R²—OH and secondly introducing compound of formula R¹—OH.

In order to minimize hydrolysis of the end product of formula (I) back to fluconazole, the end material, in whatever form, such as a filter cake, may be washed with acetone to displace the water required to hydrolyze the phosphate ester(s).

The resulting compounds may be optionally converted into a pharmaceutically acceptable salt or vice versa according to the methods known by the skilled in the art.

Further, compounds of formula (I) may be converted into each other following art-known functional group transformation reactions. For example, amino groups may be N-alkylated, nitro groups reduced to amino groups, a halo atom may be exchanged for another halo.

Pure stereochemically isomeric forms of the compounds of formula (I) may be obtained by the application of art-known procedures. Diastereomers may be separated by physical methods such as selective crystallization and chromatographic techniques, e.g., counter-current distribution, liquid chromatography and the like.

The compounds of formula (I) may be obtained as racemic mixtures of enantiomers, which can be separated from one another following art-known resolution procedures. The racemic compounds of formula (I) that are sufficiently basic or acidic may be converted into the corresponding diastereomeric salt forms by reaction with a suitable chiral acid, respectively chiral base. Said diastereomeric salt forms are subsequently separated, for example, by selective or fractional crystallization and the enantiomers are liberated therefrom by alkali or acid. An alternative manner of separating the enantiomeric forms of the compounds of formula (I) involves liquid chromatography, in particular liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound may be synthesized by stereospecific methods of preparation. These methods may advantageously employ enantiomerically pure starting materials.

The present invention further relates to intermediate compounds useful in the preparation of the compounds of formula (I); the salts, N-oxides, quaternary amines, and stereoisomers thereof. As such the present invention relates to a phosphoramidite of formula (II) 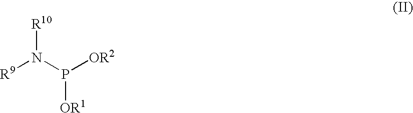
and the salts, N-oxides, quaternary amines, and stereoisomers thereof, wherein R¹, R², R⁹, and R¹⁰ are each as defined herein.

The present invention also relates to a phosphite of formula (III), as defined above. 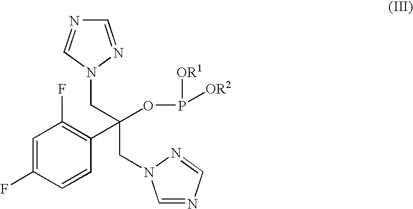
and the salts, N-oxides, quaternary amines, and stereoisomers thereof, wherein R¹ and R² are each as defined herein.

The present invention relates as well to a phosphorochloridate of formula (IV) 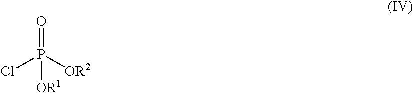
and the salts, N-oxides, quaternary amines, and stereoisomers thereof, wherein R¹ and R² are each as defined herein.

In a further aspect, the present invention relates to the use of the chemical group of formula (VI) as a promoiety 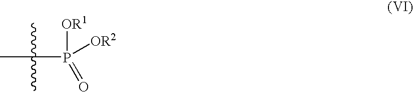
wherein R¹ and R² are each as defined above according to any one of the embodiments presented herein; and
the wavy line (depicted by [symbol] ) indicates the bond to the oxygen atom of a drug.

This promoiety is valuable in designing further prodrugs for other pharmaceutical compounds, not necessarily related to fluconazole.

The term “promoiety” refers to a chemical group, i.e. moiety, bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. Cleavage of the promoiety may proceed spontaneously, such as via a hydrolysis reaction, or may be catalyzed or induced by another agent, such as by an enzyme, by light, by acid, or by a change of or exposure to a physical or environmental parameter such as a change of temperature, pH, etc. The agent may be endogenous to the conditions of use, such as an enzyme present in the systemic circulation of a patient to which the prodrug is administered or the acidic conditions of the stomach, or the agent may be supplied exogenously.

The present invention further relates to a pharmaceutical composition comprising a pharmaceutically acceptable carrier, and as active ingredient an effective amount of the compound as defined herein or a pharmaceutically acceptable salt thereof.

The pharmaceutical composition of the present invention is parenteral, i.e., administered other than by the oral route, such as intravenously, intramuscularly, subcutaneously, intraperitoneally, intra-articularly, intralesionally, intraventricularly, by spinal injection, by intraosseous infusion, or transdermally. The pharmaceutical composition of the present invention may as well be administered intracavernously, intramyocardially, adventitially, intraturnorally, at an intracerebral portion, a wound site, tight joint spaces, or a body cavity of a human or animal.

As such, the compounds of the invention may be formulated as small-volume parenterals (SVPs) for bolus or depot injection or as large-volume parenterals (LVPs) for intravenous infusion. Alternatively, the compounds of the invention may be formulated for transdermal administration, through the use of skin patches.

The parenteral formulations may take such forms as suspensions, solutions or emulsions in oily or aqueous vehicles, and may contain formulatory agents such as pH modifiers, complexing, isotonizing, suspending, stabilizing, or dispersing agents. In general, those excipients typically used for intravenous injections may be used in the formulations of the present invention as long as the formulation presents an appropriate viscosity, a reduced injection volume, and an acceptable pH range.

Methods of preparing various pharmaceutical compositions with a certain amount of active ingredient are known, or will be apparent in light of this disclosure, to those skilled in this art. For examples of methods of preparing pharmaceutical compositions, see (Remington; the science and practice of pharmacy, Lippincott Williams & Wilkins, 21st Ed, 2006).

The unit dosage forms are prepared by admixing a compound of the invention or a pharmaceutically acceptable salt thereof and a sterile vehicle. The compound, depending on the vehicle and concentration used, can be either suspended or dissolved in the vehicle. In preparing solutions, the compound can be dissolved for injection and filter sterilized before filling into a suitable vial or ampoule and sealing. Advantageously, adjuvants such as local anaesthetic preservatives and buffering agents are dissolved in the vehicle. To enhance the stability, the composition can be frozen after filling into the vial and the water removed under vacuum. The pharmaceutical composition will be then presented in powder form for later constitution with a suitable vehicle, e.g. sterile pyrogen-free water before use. Suspensions are prepared in substantially the same manner, except that the compound is suspended in the vehicle instead of being dissolved and sterilization cannot be accomplished by filtration. A surfactant or wetting agent may be included in the composition to facilitate a uniform distribution of the compound.

Formulations for injection may be presented in unit dosage form e.g. in ampoules or in multidose Containers.

For purposes of transdermal (e.g., topical) administration, dilute sterile, aqueous or partially aqueous solutions (usually in about 0.1% to 5% concentration), otherwise similar to the above parenteral solutions, are prepared.

Therapeutically effective doses of the compounds of the present invention required to prevent or to treat the medical condition are readily ascertained by one of ordinary skill in the art using preclinical and clinical approaches familiar to the medicinal arts. The dose of the compound or a pharmaceutically acceptable salts thereof to be administered depends on the individual case and, as customary, is to be adapted to the conditions of the individual case for an optimum effect. Thus it depends, of course, on the frequency of administration and on the potency and duration of action of the compound employed in each case for therapy or prophylaxis, but also on the nature and severity of the disease and symptoms, and on the sex, age, weight co-medication and individual responsiveness of the subject to be treated and on whether the therapy is acute or prophylactic. The percentage of drug present in the formulation is also a factor. Doses may be adapted in function of weight and for pediatric applications. An example of an effective dose for injection of a formulation of the present invention is from about 0.1 ml to about 5 ml injected once every 1, 2, 3, 4, 5, 6 months, or injected once every 1, 2, 3 or 4 weeks. Preferably, the dose for injection is about 2.5 ml or less, for example from about 1 ml to about 2.5 ml.

The compounds of the present invention are useful because they possess pharmacological activity in animals, including humans. In particular, the compounds are useful in the treatment or prevention of fungal infections, including yeast infections. For example, they are useful in treating topical fungal infections in man caused by, among other organisms, species of Candida, Trichophyton, Microsporum or Epidermophyton, or in mucosal infections caused by Candida albicans (e.g. thrush and vaginal candidiasis). They can also be used in the treatment of systemic fungal infections caused by, for example, species of Candida (e.g. Candida albicans), Cryptococcus neofonnans, Aspergillus flavus, Aspergillus fumigatus, Coccididides, Paracoccidiodes, Histoplasma, or Blastomyces.

Furthermore, the compounds of the present invention may be used to treat mycotic peritonitis among deep-seated mycosis agents, in addition to the following diseases for which fluconazole is indicated: fungemia, respiratory tract mycosis, digestive tract mycosis, urinary tract mycosis, mycotic meningitis, cryptococcal meningitis, onychomycosis, cryptococcosis, coccidiomycosis, and the like.

The compounds of the present invention may also be used as a prophylactic agent of candidiasis in immunocompromised patients, such as those patients with hematologic cancers, organ transplants, AIDS, or in elder or pediatric populations.

The compounds of the present invention, pharmaceutically acceptable salts thereof, or any subgroup thereof may therefore be used as medicines. Said use as a medicine or method of treatment comprises the systemic administration to infected subjects or to subjects susceptible to fungal infections, including yeasts, of an amount effective to combat the conditions associated with the fungal infection, in particular Candida infection.

The present invention also relates to the use of the present compounds, pharmaceutically acceptable salts thereof, or any subgroup thereof for the manufacture of a medicament for the prevention or treatment of fungal injections. In other words, the present invention further relates to the compounds of formula (I), pharmaceutically acceptable salts thereof, or any subgroup thereof, for use in the prevention or treatment of fungal infections

The present invention furthermore relates to a method of prevention or treatment of fungal infections, said method comprising the administration of an effective amount of a compound of formula (I), a pharmaceutically acceptable salt thereof, or of a compound of any of the subgroups of compounds of formula (I), as specified herein, to a patient in need of such prevention or treatment.

The present invention also relates to a method of improving the lipophilicity of fluconazole, which comprises converting said fluconazole into a compound of formula (I), a pharmaceutically acceptable salt thereof, or any subgroup thereof.

In addition, the present invention relates to a method of extending the release of fluconazole, which comprises converting fluconazole into a compound of formula (I), a pharmaceutically acceptable salt thereof, or any subgroup thereof.

The following examples are intended to illustrate the present invention and not to limit it thereto.

### EXAMPLES

### Example 1

### Preparation of 2-(2,4-difluorophenyl)-1,3-di(1H-1,2,4-triazol-1-yl)propan-2-yl 2-(trimethylammonio)-ethyl phosphate

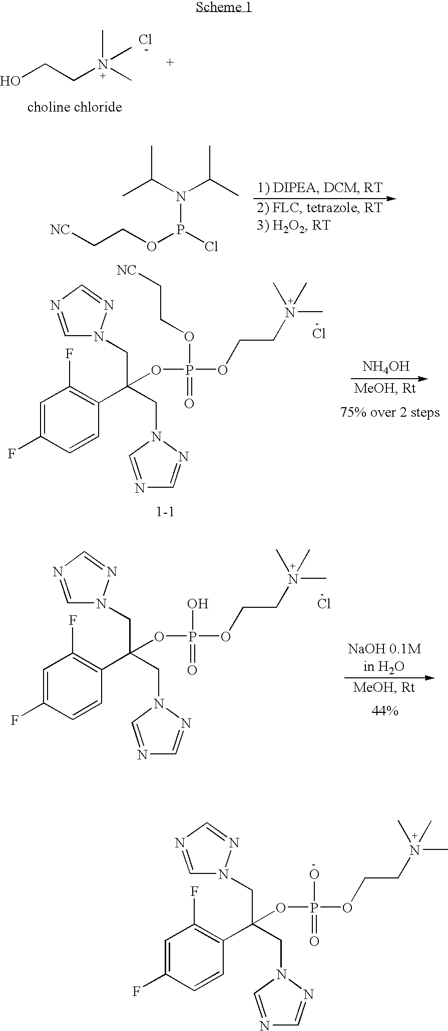

As shown in Scheme 1 above, the reaction of choline chloride with the phosphorylating reagent cyanoethyl N,N-diisopropylchlorophosphoramidite, in the presence of N,N-diisopropylethylamine (Hünig's base) in dry dichloromethane at room temperature, subsequent coupling with fluconazole (FLC) in the presence of tetrazole followed by oxidation with 30% aqueous hydrogen peroxide afforded in a one-pot process the crude fosfluconazole derivative 1.1. The cyanoethyl protecting group was cleaved using ammonium hydroxide in methanol at room temperature (75% yield over two steps). The resulting phosphate diester was further converted into the corresponding ammonium phosphate zwitterionic salt by reaction with aqueous sodium hydroxide (0.1 M) in methanol, in 44% yield after chromatography. The final product, 2-(2,4-difluorophenyl)-1,3-di(1H-1,2,4-triazol-1-yl)propan -2-yl 2-(trimethylammonio)-ethyl phosphate, was characterized by proton nuclear magnetic resonance and mass spectrometry as follows:
¹H NMR (400 MHz, DMSO-d₆): δ 9.00 (s, 2H), 7.60 (s, 2H), 7.07 (m, 1H), 6.86 (m, 1H), 6.61 (m, 1H), 5.50 and 4.91 (AB system, J_{AB}=14.4 Hz, 4H), 4.22 (m, 2H), 3.59 (m, 2H), 3.15 (s, 9H)
MS (ESI): 472 (M+H)

### Example 2

### Preparation of sodium 2-(decanoyloxy)ethyl 2-(2,4-difluorophenyl)-1,3-di(1H -1,2,4-triazol-1-yl)propan-2-yl phosphate

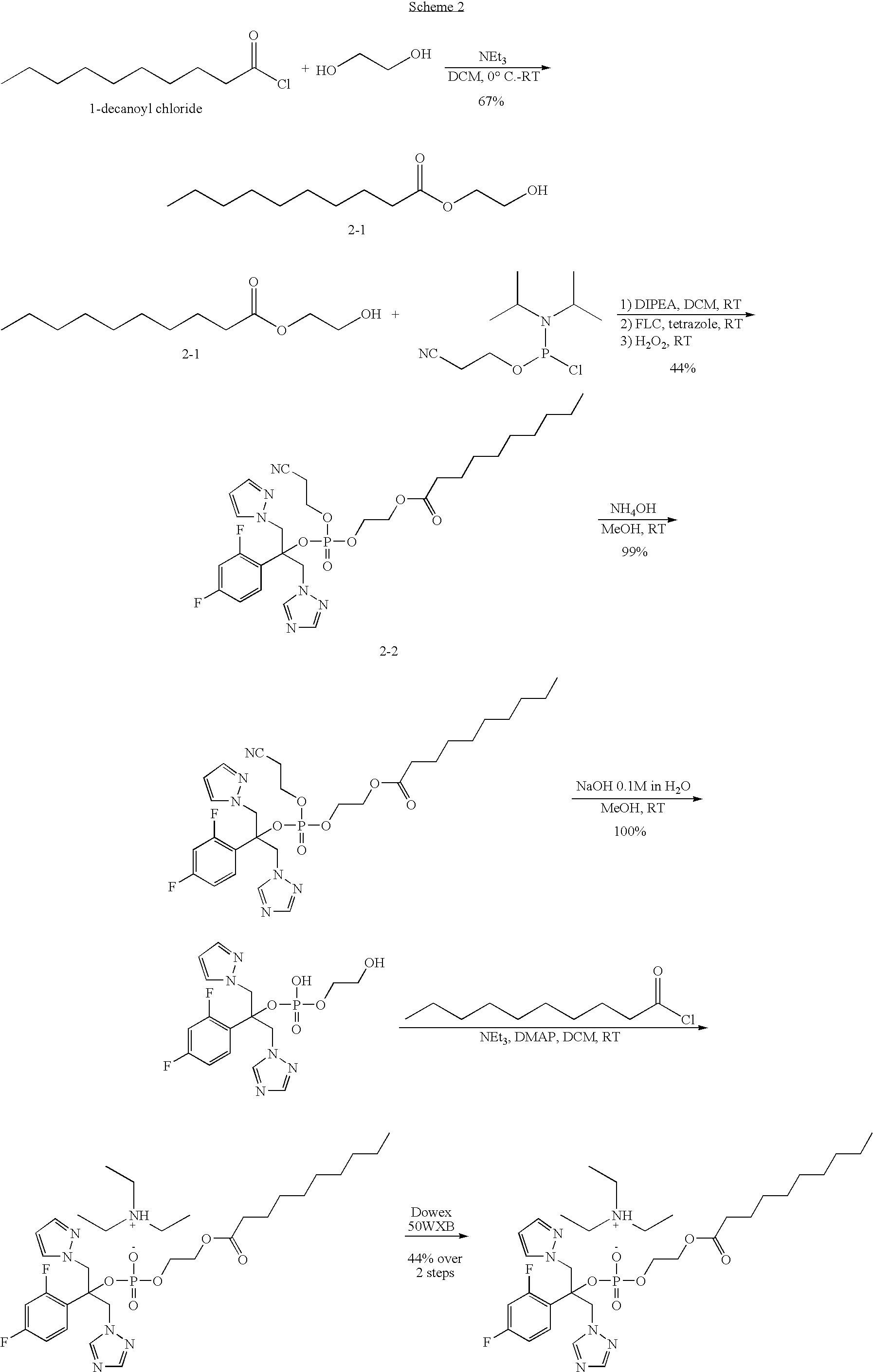

As shown in Scheme 2 above, the reaction between 1-decanoyl chloride and ethylene glycol in the presence of triethylamine in dry dichloromethane at room temperature gave the hydroxyester 2.1 in 67% yield. The reaction of 2.1 with the phosphorylating reagent cyanoethyl N,N-diisopropylchlorophosphoramidite, in the presence of N,N-diisopropylethylamine (Hünig's base) in dry dichloromethane at room temperature, subsequent coupling with fluconazole (FLC) in the presence of tetrazole followed by oxidation with 30% aqueous hydrogen peroxide afforded in a one-pot process the fosfluconazole derivative 2.2 in 44% yield after chromatography. The cyanoethyl protecting group was cleaved using ammonium hydroxide in methanol at room temperature in 99% yield. The resulting phosphate diester was further submitted to saponification with aqueous sodium hydroxide (0.1 M) in methanol (100% yield) and acylation with n-nonanoyl chloride and triethylamine, in dichloromethane and in the presence of 4-dimethylaminopyridine, to afford the triethylammonium phosphate salt. After chromatography, a cation exchange using Dowex 50WX8 gave the corresponding sodium salt, in 44% yield over two steps. The final product, sodium 2-(decanoyloxy)ethyl 2-(2,4-difluorophenyl)-1,3-di(1H-1,2,4 -triazol-1-yl)propan-2-yl phosphate, was characterized by proton nuclear magnetic resonance and mass spectrometry as follows:
¹H NMR (400 MHz, DMSO-d₆): δ 9.00 (s, 2H), 7.55 (s, 2H), 7.05 (m, 1H), 6.87 (m, 1H), 6.59 (m, 1H), 5.55 and 4.87 (AB system, J_{AB}=14.4 Hz, 4H), 4.14 (m, 2H), 3.98 (m, 2H), 2.27 (m, 2H), 1.47 (m, 2H), 1.21 (m, 12H), 0.84 (t,J=6.4 Hz, 3H)
MS (ESI): 583 (M-H—Na)

### Example 3

### Preparation of sodium 2-(2,4-difluorophenyl)-1,3-di(1H-1,2,4-triazol-1-yl)propan-2-yl 2-(nonyloxy)ethyl phosphate

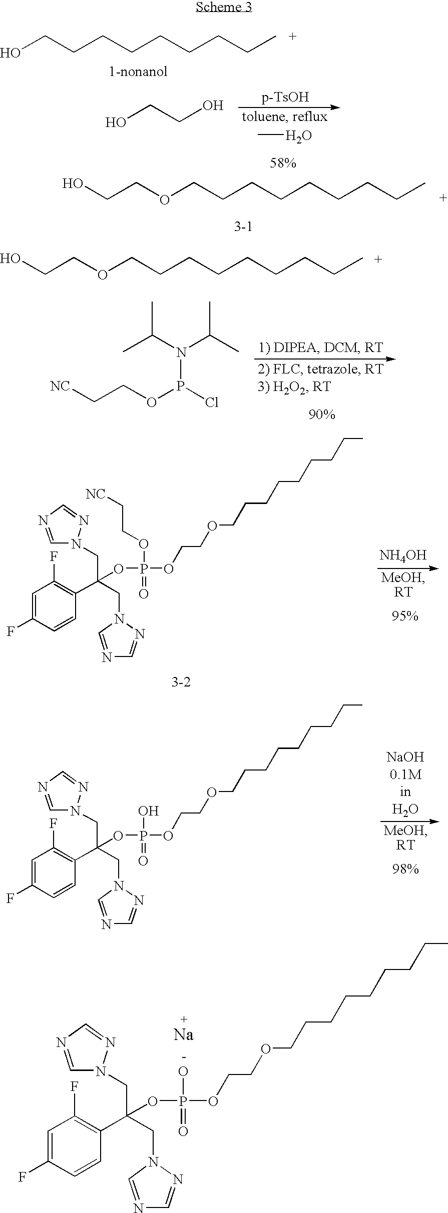

As shown in Scheme 3 above, the reaction between 1-nonanol and ethylene glycol in refluxing toluene in the presence of p-toluenesulfonic acid using Dean-Stark conditions gave the hydroxyether 3.1 in 58% yield. The reaction of 3.1 with the phosphorylating reagent cyanoethyl N,N-diisopropylchlorophosphoramidite, in the presence of N,N-diisopropylethylamine (Hünig's base) in dry dichloromethane at room temperature, subsequent coupling with fluconazole (FLC) in the presence of tetrazole followed by oxidation with 30% aqueous hydrogen peroxide afforded in a one-pot process the fosfluconazole derivative 3.2 in 90% yield after chromatography. The cyanoethyl protecting group was cleaved using ammonium hydroxide in methanol at room temperature in 95% yield. The resulting phosphate diester was converted into the sodium salt by reaction with aqueous sodium hydroxide (0.1 M) in methanol in 98% yield. The final product, sodium 2-(2,4-difluorophenyl)-1,3-di(1H-1,2,4-triazol-1-yl)propan-2-yl 2-(nonyloxy)ethyl phosphate, was characterized by proton nuclear magnetic resonance and mass spectrometry as follows:
¹H NMR (400 MHz, DMSO-d₆): δ 9.01 (s, 2H), 7.55 (s, 2H), 7.05 (m, 1H), 6.88 (m, 1H), 6.60 (m, 1H), 5.55 and 4.87 (AB system, J_{AB}=14.4 Hz, 4H), 3.89 (m, 2H), 3.49 (m, 2H), 3.36 (m, 2H), 1.46 (m, 2H), 1.22 (m, 12H), 0.84 (t,J=6.4 Hz, 3H)
MS (ESI): 555 (M-H—Na)

### Example 4

### Preparation of sodium 2,3-bis(decanoyloxy)propyl 2-(2,4-difluorophenyl)-1,3-di(1H-1,2,4-triazol-1-yl)propan-2-yl phosphate

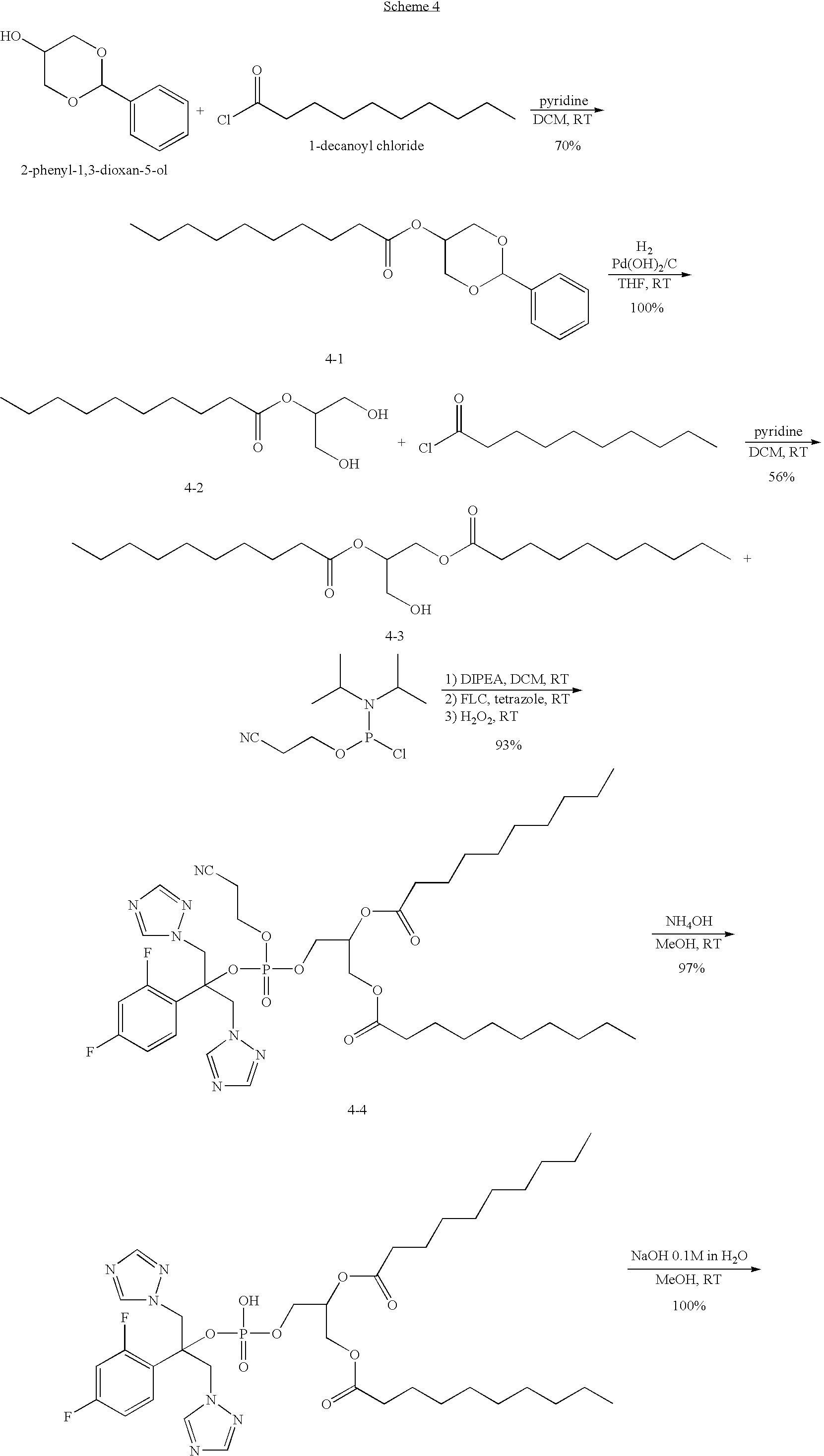

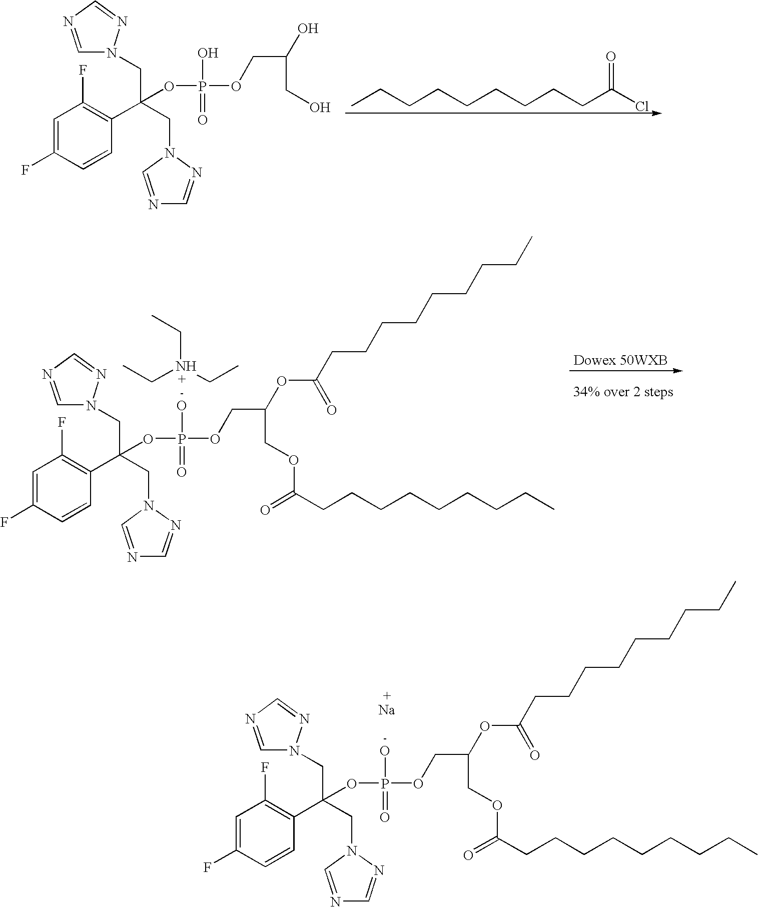

As shown in Scheme 4 above, the reaction between 2-phenyl-1,3-dioxan-5-ol and 1-decanoyl chloride in the presence of pyridine in dry dichloromethane at room temperature gave the ester 4.1 in 70% yield. The compound 4.1 was then submitted to hydrogenation using palladium hydroxide on carbon and an atmospheric pressure of hydrogen to afford the diol 4-2 quantitatively. Subsequent mono-acylation of 4-2 with 1-decanoyl chloride gave the primary alcohol 4-3 in 56% yield. The reaction of compound of 4.3 with the phosphorylating reagent cyanoethyl N,N-diisopropylchlorophosphoramidite, in the presence of N,N-diisopropylethylamine (Hünig's base) in dry dichloromethane at room temperature, subsequent coupling with fluconazole (FLC) in the presence of tetrazole followed by oxidation with 30% aqueous hydrogen peroxide afforded in a one-pot process the fosfluconazole derivative 4.4 in 93% yield after chromatography. The cyanoethyl protecting group was cleaved using ammonium hydroxide in methanol at room temperature in 97% yield. The resulting phosphate diester was further submitted to saponification with aqueous sodium hydroxide (0.1 M) in methanol (100% yield) and acylation with n-nonanoyl chloride and triethylamine, in dichloromethane and in the presence of 4-dimethylaminopyridine, to afford the triethylammonium phosphate salt. After chromatography, a cation exchange using Dowex 50WX8 gave the corresponding sodium salt, in 34% yield over two steps. The final product, sodium 2,3-bis(decanoyloxy)propyl 2-(2,4-difluorophenyl)-1,3-di(1H-1,2,4-triazol-1-yl)propan-2-yl phosphate, was characterized by proton nuclear magnetic resonance and mass spectrometry as follows:

¹H NMR (400 MHz, DMSO-d₆): δ 8.98 (d, 2H), 7.55 (s, 2H), 7.06 (m, 1H), 6.88 (m, 1H), 6.58 (m, 1H), 5.52 and 4.88 (AB system, J_{AB}=14.4 Hz, 4H), 5.11 (m, X of ABX, 1H), 4.29 and 4.11 (AB of ABX, J_{AB}=12.0 Hz, J_{AX}=3.3 Hz, J_{BX}=6.7 Hz, 2H), 3.93 (m, 2H), 2.26 (m, 4H), 1.48 (m, 4H), 1.22 (m, 24H), 0.84 (t,J=5.8 Hz, 6H)
MS (ESI): 767 (M-H—Na)

### Example 5

### Preparation of 2-(2,4-difluorophenyl)-1,3-di(1H-1,2,4-triazol-1-yl)propan-2-yl ethyl (3R,8R,9S,10R,13S,14S,17R)-10,13,17-trimethyl-17-((R)-6-methylheptan-2-yl)-2,3,4,7,8,9,10,11,12,13,14,15,16,17-tetradecahydro-1H-cyclopenta[a]phenanthren-3-yl phosphate

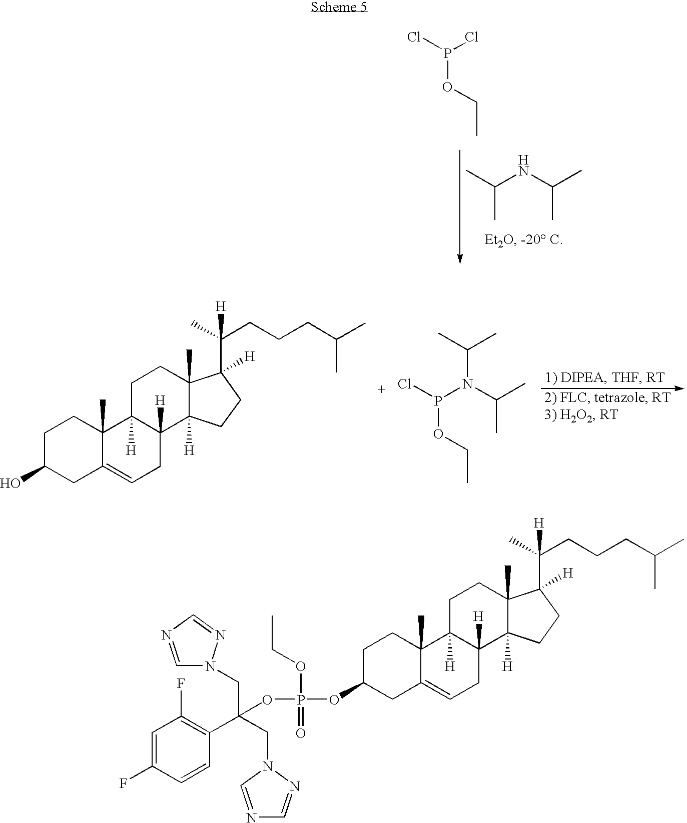

As shown in Scheme 5 above, the reaction of cholesterol with the freshly prepared phosphorylating reagent ethyl N,N-diisopropylchlorophosphoramidite, in the presence of N,N-diisopropylethylamine (Hünig's base) in dry tetrahydrofuran at room temperature, subsequent coupling with fluconazole (FLC) in the presence of tetrazole followed by oxidation with 30% aqueous hydrogen peroxide afforded in a one-pot process the desired fosfluconazole derivative in 76% yield after chromatography. The resulting product was characterized by proton nuclear magnetic resonance and mass spectrometry as follows:
¹H NMR (400 MHz, CDCl₃): δ 8.41 (m, 2H), 7.77 (m, 2H), 7.07 (m, 1H), 6.84 (m, 1H), 6.72 (m, 1H), 5.33 (m, 1H), 5.16 (m, 4H), 4.14 (m, 1H), 4.02 (m, 2H), 2.28 (m, 2H), 1.96 (m, 2H), 1.88-0.99 (m, 27H), 0.96 (s, 3H), 0.90 (d, J=10.4 Hz, 3H), 0.84 and 0.82 (dd, J=1.5 Hz, 6H), 0.64 (s, 3H)
MS (APCI): 783 (M)
White powder

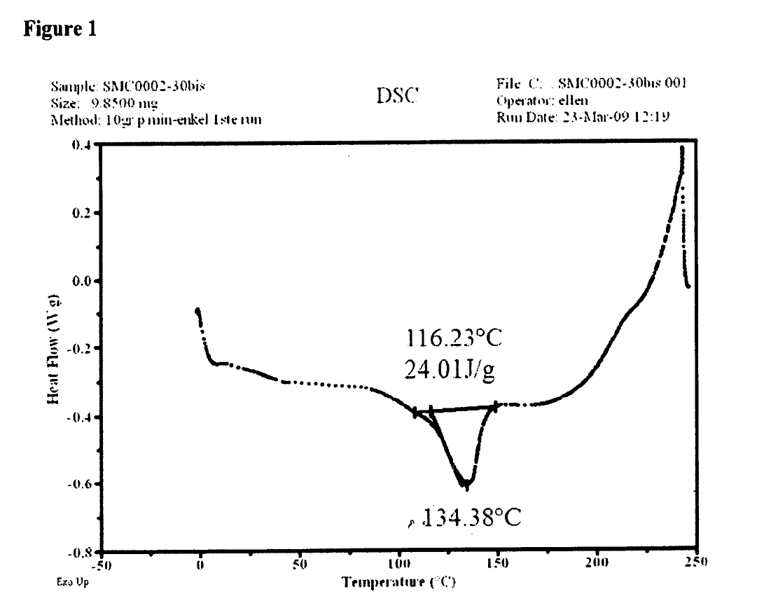

A melting point DSC characterization of the product is presented in FIG. 1.

### Example 6

### Preparation of 2-((2-(2,4-difluorophenyl)-1,3-di(1H-1,2,4-triazol-1-yl)propan -2-yloxy)(ethoxy)phosphoryloxy)ethyl(3S,8R,9S,10R,13S,14S,17R)-10,13,17 -trimethyl-17 -((R)-6-methylheptan-2-yl)-2,3,4,7,8,9,10,11,12,13,14,15,16,17-tetradecahydro-1H -cyclopenta[a]phenanthren-3-yl succinate

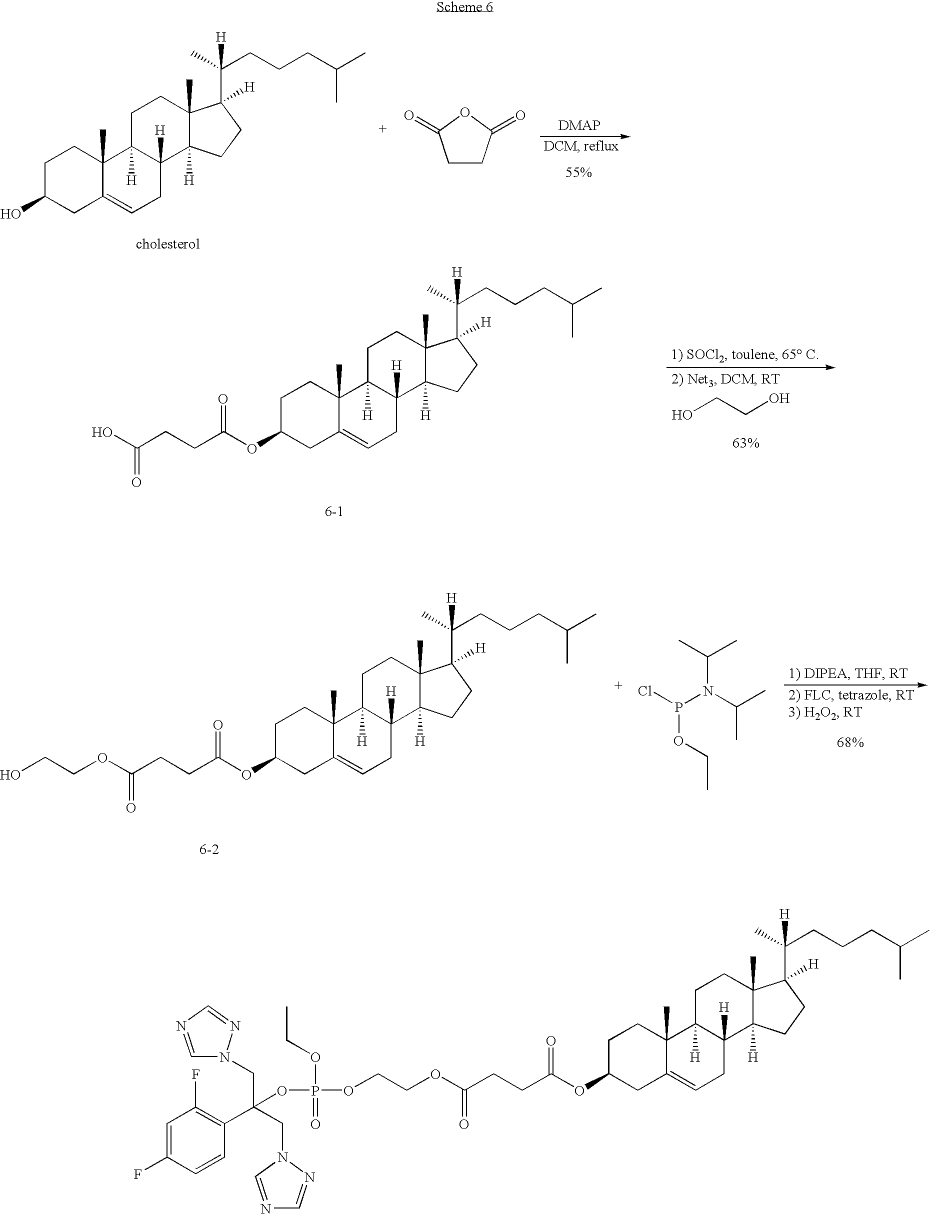

As shown in Scheme 6 above, cholesterol was reacted with succinic anhydride in the presence of 4-dimethylaminopyridine in refluxing dichloromethane to afford in 55% yield the carboxylic acid 6.1, which was converted into its acid chloride with thionyl chloride in toluene at 65° C. and further reacted with ethylene glycol in the presence of triethylamine in dichloromethane at room temperature to give derivative 6.2 in 63% yield. The reaction of 6.2 with the freshly prepared phosphorylating reagent ethyl N,N-diisopropylchlorophosphoramidite, in the presence of N,N-diisopropylethylamine (Hünig's base) in dry tetrahydrofuran at room temperature, subsequent coupling with fluconazole (FLC) in the presence of tetrazole followed by oxidation with 30% aqueous hydrogen peroxide afforded in a one-pot process the desired fosfluconazole derivative in 68% yield after chromatography. The resulting product was characterized by proton nuclear magnetic resonance and mass spectrometry as follows:
¹H NMR (400 MHz, CDCl₃): δ 8.39 (d, J=14.9 Hz, 2H), 7.81 (s, 2H), 7.11 (m, 1H), 6.88 (m, 1H), 6.77 (m, 1H), 5.35 (m, 1H), 5.18 (m, 4H), 4.59 (m, 1H), 4.24 (m, 2H), 4.21-4.01 (m, 4H), 2.61 (m, 4H), 2.30 (m, 2H), 1.98 (m, 2H), 1.83 (m, 2H), 1.62-1.03 (m, 25H), 1.00 (s, 3H), 0.90 (d, J=6.6 Hz, 3H), 0.86 and 0.84 (dd, J=1.8 Hz, 6H), 0.67 (s, 3H)
MS (APCI): 928 (M)
White powder

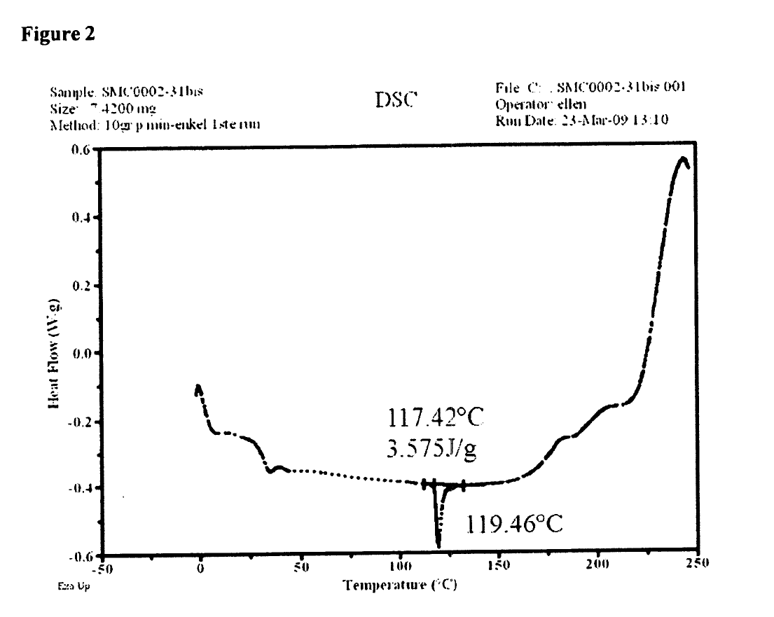

A melting point DSC characterization of the product is presented in FIG. 2.

### Example 7

### Solubility of fosfluconazole derivatives in pharmaceutical solvents

Table 2 provides the solubility data at 25° C., expressed in mg/mL, unless otherwise indicated, of the final fosfluconazole derivatives produced and characterized in examples 1-6 and 14, respectively. The solubility determination method used was as follows: a suspension of 6 mg of the tested fosfluconazole derivative in 500 μl of the applicable pharmaceutical solvent was rotatively shaken for 24 hours at 800 rpm at 25° C. The saturated fosfluconazole derivative solution was filtered (0.45 μm) and 150 μl of the filtrate was diluted in dimethylsulfoxide (50 μl DMSO). This solution was assayed, each assayed being carried out in three-fold, by measurement with Liquid Chromatography-Mass Spectrometry (LCMS, 1 μl and 10 μl injection). Standards were prepared by dissolving the corresponding fosfluconazole derivative in DMSO (1 mg/ml). To determine the solubility, four aliquots (0.5, 1, 2, 4 μl) of the standard solution were injected using the same LCMS conditions as for the above exemplary samples.

TABLE 2

| | Compound of Example: | | | | | | |
|---|---|---|---|---|---|---|---|
| Pharmaceutical solvent | 1 | 2 | 3 | 4 | 5 | 6 | 14 |
| H₂O (pH = 2) | >10 | 6.9 | >10 | <1 | <1 mg/L | <1 mg/L | >10 |
| H₂O (pH = 7) | 6.8 | >10 | >10 | 9.9 | <1 mg/L | <1 mg/L | >10 |
| H₂O (pH = 10) | 7.5 | >10 | >10 | >10 | <1 mg/L | <1 mg/L | >10 |
| H₂O/Kleptose HPB ^{a} (60/40) | 4.2 | >10 | >10 | >10 | 0.5 | 0.6 | >10 |
| H₂O/Vitamin E TPGS (90/10) | 7.4 | >10 | >10 | >10 | 1.4 | 6.2 | >10 |
| H₂O/Cremophor RH 40 ^{b} (80/20) | >10 | >10 | >10 | >10 | 0.9 | <1 mg/L | >10 |
| H₂O/polysorbate 80 (80/20) | 8.3 | >10 | >10 | >10 | 4.5 | >10 | >10 |
| PEG 400 | >10 | >10 | >10 | 9.0 | 4.1 | >10 | >10 |
| Miglyol 812 ^{c} | 0.4 | 5.0 | 2.6 | >10 | 9.7 | >10 | 1.2 |

^{a} hydroxypropyl-beta-cyclodextrin-HPBCD
^{b} an emulsifying agent obtained by reacting 45 moles of ethylene oxide with 1 mole of hydrogenated castor oil, commercially available from BASF AG (Germany).
^{c} a caprylic/capric acid triglyceride, commercially available from SASOL GmbH (Germany).

TABLE 3

| | Compound of Example: | |
|---|---|---|
| Pharmaceutical solvent | 5 | 6 |
| N-methylpyrrolidone | 226.7 (88.6) | 226.7 (74.8) |
| Miglyol 812 | 12.0 (4.7) | 200.0 (66.0) |
| Sesame oil (LCT) | 10.0 (3.9) | 23.3 (7.7) |
| Benzylalcohol | 226.7 (88.6 | 226.7 (748) |
| Benzylbenzoate 90.0 (35,2) | 226.7 (74,8) | |
| Ethylbenzoate | 133.0 (52.1) | 226.7 (74.8) |

The values within brackets in Table 3 correspond to the calculated fluconazole concentrations taking into account the molecular weights of the relevant fosfluconazole prodrug and fluconazole itself.

### Example 8

### Chemical stability of the fosfluconazole derivative 2-(2,4-difluorophenyl)-1,3-di(1H-1,2,4-triazol-1-yl)propan-2-yl 2-(trimethylammonio)-ethyl phosphate in pharmaceutical solvents

Table 4 provides the chemical stability data in some pharmaceutical solvents, expressed as the weight percentage (% w/w) of the final fosfluconazole derivative produced and characterized in example 1 that remained in solution after a certain period of time. The test was carried out twice, and the remaining weight % was determined by Liquid Chromatography-Mass Spectrometry (LCMS).

TABLE 4

| Pharmaceutical solvent | 4 hours | 1 day | 2 days | 7 days | 14 days |
|---|---|---|---|---|---|
| H₂O (pH = 2) | 99.9 | 100.0 | 99.4 | 99.4 | 99.2 |
| H₂O (pH = 7) | 99.6 | 99.6 | 54.7 | 99.6 | 99.6 |
| H₂O (pH = 10) | 99.8 | 100.0 | 99.7 | 100.0 | 00.0 |
| H₂O/Kleptose HPB (60/40) | 99.3 | 98.8 | 98.8 | 98.6 | 99.1 |
| PEG 400 | 99.8 | 100.0 | 99.8 | 99.9 | 100.0 |

Table 4 shows that at most 1% of the fosfluconazole derivative did not remain in the relevant aqueous or hydroxypropyl-β-cyclodextrin or polyethylene glycol solution after 14 days.

### Example 9

### Chemical stability of the fosfluconazole derivative sodium 2-(decanoyloxy)ethyl 2-(2,4-difluorophenyl)-1,3-di(1H-1,2,4-triazol-1-yl)propan-2-yl phosphate in pharmaceutical solvents

Table 5 provides the chemical stability data in some pharmaceutical solvents at 25° C., expressed as the weight percentage (% w/w) of the final fosfluconazole derivative produced and characterized in example 2 that remained in solution after a certain period of time. The test was carried out twice, and the remaining weight % was determined by Liquid Chromatography-Mass Spectrometry (LCMS).

TABLE 5

| Pharmaceutical solvent | 4 hours | 1 day | 2 days | 7 days | 14 days |
|---|---|---|---|---|---|
| H₂O (pH = 2) | 97.8 | 97.7 | 96.8 | 96.7 | 100.0 |
| H₂O (pH = 7) | 97.8 | 97.7 | 98.0 | 97.8 | 97.9 |
| H₂O (pH = 10) | 94.6 | 98.0 | 96.8 | 96.8 | 99.2 |
| H₂O/Kleptose HPB (60/40) | 98.0 | 98.0 | 98.2 | 97.7 | 98.0 |
| PEG 400 | 97.7 | 97.9 | 98.1 | 98.0 | 98.7 |

Table 5 shows that at most about 2% of the fosfluconazole derivative did not remain in the relevant aqueous or hydroxypropyl-β-cyclodextrin or polyethylene glycol solution after 14 days.

### Example 10

### Chemical stability of the fosfluconazole derivative sodium 2-(2,4-difluorophenyl)-1,3-di(1H-1,2,4-triazol-1-yl)propan-2-yl 2-(nonyloxy)ethyl phosphate in pharmaceutical solvents

Table 6 provides the chemical stability data in some pharmaceutical solvents at 25° C., expressed as the weight percentage (% w/w) of the final fosfluconazole derivative produced and characterized in example 3 that remained in solution after a certain period of time. The test was carried out twice, and the remaining weight % was determined by Liqauid Chromatography-Mass Spectrometry (LCMS).

TABLE 6

| Pharmaceutical solvent | 4 hours | 1 day | 2 days | 7 days | 14 days |
|---|---|---|---|---|---|
| H₂O (pH = 2) | 94.8 | 94.6 | 95.0 | 95.0 | 95.0 |
| H₂O (pH = 7) | 94.8 | 94.7 | 95.0 | 94.8 | 95.1 |
| H₂O (pH = 10) | 94.8 | 94.8 | 95.0 | 95.0 | 95.2 |
| H₂O/Kleptose HPB (60/40) | 95.0 | 94.9 | 94.7 | 95.0 | 95.2 |
| PEG 400 | 95.0 | 94.9 | 95.0 | 95.0 | 95.1 |

Table 6 shows that at most about 5% of the fosfluconazole derivative did not remain in the relevant aqueous or hydroxypropyl-β-cyclodextrin or polyethylene glycol solution after 14 days.

### Example 11

### Chemical stability of the fosfluconazole derivative sodium 2,3-bis(decanoyloxy)propyl 2-(2,4-difluorophenyl)-1,3-di(1H-1,2,4-triazol-1-yl)propan-2-yl phosphatein pharmaceutical solvents

Table 7 provides the chemical stability data in some pharmaceutical solvents, expressed as the weight percentage (% w/w) of the final fosfluconazole derivative produced and characterized in example 4 that remained in solution after a certain period of time. The test was carried out twice, and the remaining weight % was determined by Liquid Chromatography-Mass Spectrometry (LCMS).

TABLE 7

| Pharmaceutical solvent | 4 hours | 1 day | 20 days | 7 days | 14 days |
|---|---|---|---|---|---|
| H₂O (pH = 7) | 95.2 | 95.3 | 95.5 | 95.6 | 95.4 |
| H₂O (pH = 10) | 45.5 | 44.8 | 41.3 | 39.4 | 37.1 |
| H₂O/Kleptose HPB (60/40) | 95.1 | 95.3 | 95.3 | 94.2 | 87.7 |
| PEG 400 | 96.8 | 95.9 | 96.7 | 96.6 | 97.3 |

Table 7 shows that, except in water at pH 10, at most about 12% of the fosfluconazole derivative did not remain in the relevant aqueous or hydroxypropyl-β-cyclodextrin or polyethylene glycol solution after 14 days.

### Example 12

### Chemical stability of the fosfluconazole derivative 2-(2,4-difluorophenyl)-1,3 -di(1H-1,2,4-triazol-1-yl)propan-2-yl ethyl(3R,8R,9S,10R,13S,14S,17R) -10,13,17-trimethyl-17-((R)-6-methylheptan-2-yl)-2,3,4,7,8,9,10,11,12,13,14,15,16,17-tetradecahydro-1H -cyclopenta[a]phenanthren-3-yl phosphate in pharmaceutical solvents

Table 8 provides the chemical stability data in some pharmaceutical solvents, expressed as the weight percentage (% w/w) of the final fosfluconazole derivative produced and characterized in example 5 that remained in solution after a certain period of time. The test was carried out twice, and the remaining weight % was determined by Liquid Chromatography-Mass Spectrometry (LCMS).

TABLE 8

| | 2 | 4 | | 2 | 7 | 14 |
|---|---|---|---|---|---|---|
| Pharmaceutical solvents | hours | hours | 1 day | days | days | days |
| H₂O (pH = 2) | 74.5 | 76.9 | 76.9 | 76.9 | 0.0 | 0.0 |
| H₂O (pH = 7) | 100.0 | 100.0 | 100.0 | 100.0 | 100.0 | 100.0 |
| H₂O (pH = 10) | 100.0 | 100.0 | 100.0 | 100.0 | 100.0 | 100.0 |
| H₂O/Kleptose HPB (60/40) | 92.0 | 92.0 | 92.0 | 92.0 | 92.0 | 92.0 |
| PEG 400 | 97.7 | 97.7 | 97.7 | 97.7 | 97.7 | 97.7 |

Table 8 shows that, except in water at pH 2, at most about 8% of the fosfluconazole derivative did not remain in hydroxypropyl-β-cyclodextrin solution after 14 days.

### Example 13

### Chemical stability of the fosfluconazole derivative 2-((2-(2,4-difluorophenyl) -1,3-di(1H-1,2,4-triazol-1-yl)propan-2-yloxy)(ethoxy)phosphoryloxy)ethyl (3R,8R,9S,10R,13S,14S,17R)-10,13,17-trimethyl-17-((R)-6-methylheptan-2-yl) -2,3,4,7,8,9,10,11,12,13,14,15,16,17-tetradecahydro-1H-cyclopenta[a]phenanthren-3-yl succinatein pharmaceutical solvents

Table 9 provides the chemical stability data in some pharmaceutical solvents, expressed as the weight percentage (% w/w) of the final fosfluconazole derivative produced and characterized in example 6 that remained in solution after a certain period of time. The test was carried out twice, and the remaining weight % was determined by Liquid Chromatography-Mass Spectrometry (LCMS).

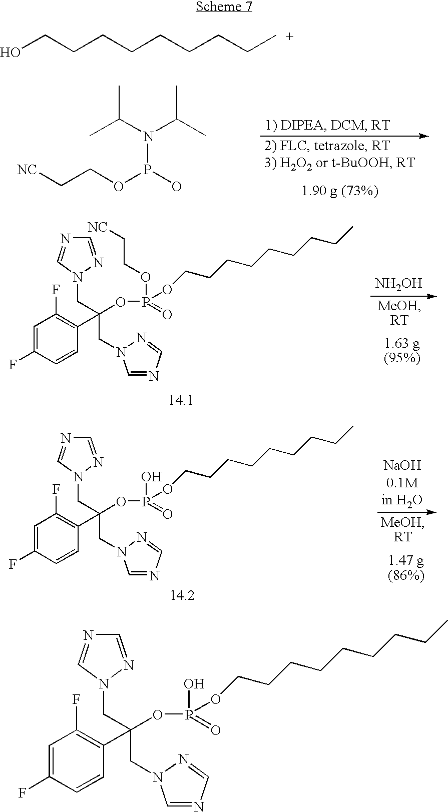

Table 9 shows that, except in water at pH 2, at most about 13% of the fosfluconazole derivative did not remain in hydroxypropyl-β-cyclodextrin solution after 14 days.

### Example 14

### Preparation of sodium 2-(2,4-difluorophenyl)-1,3-di(1H-1,2,4-triazol-1-yl)propan-2-yl nonyl phosphate

TABLE 10

| Pharmaceutical solvents | 4 hours | 1 day | 2 days | 7 days | 14 days |
|---|---|---|---|---|---|
| H₂O (pH = 2) | 99.0 | 99.1 | 99.2 | 99.2 | 99.6 |
| H₂O (pH = 7) | 99.1 | 99.3 | 99.2 | 99.2 | 99.4 |
| H₂O (pH = 10) | 99.3 | 99.3 | 99.4 | 99.3 | 99.9 |
| H₂O/Kleptose HPB (60/40) | 99.5 | 99.3 | 99.2 | 99.3 | 100.0 |
| PEG 400 | 99.3 | 99.3 | 99.4 | 99.4 | 100.0 |

As shown on scheme 7 above, nonan-1-ol was reacted in a first step with the phosphoramide derivative represented by the structural formula (C₃H₇)₂N—P(Cl)—O—(CH₂)₂—CN, i.e. (3-[chloro-[di(propan-2-yl)amino]phosphanyl]oxypropanenitrile), at room temperature in dichloromethane as a solvent and in the presence of N,N-diisopropylethylamine (Hünig's base), and then adding fluconazole at room temperature in the presence of tetrazole, and finally adding a hydrogen peroxide aqueous solution or a tert-butyl peroxide aqueous solution at room temperature, thus resulting into a fluconazole derivative 14.1. The reaction yield of this step was 73%.

Then the fluconazole derivative 14.1 was converted, by reaction at room temperature with aqueous ammonia in methanol as a solvent, into a fluconazole derivative 14.2. The reaction yield in this step was 95%. In a further step the latter fluconazole derivative 14.2 was transformed into the corresponding ammonium phosphate zwitterionic salt by reaction at room temperature with aqueous sodium hydroxide (0.1 M) in methanol as a solvent. The reaction yield in this step was 86%. The final product, sodium 2-(2,4-difluorophenyl)-1,3-di(1H-1,2,4-triazol-1-yl)propan-2-yl nonyl phosphate was characterised by proton nuclear magnetic resonance and mass spectrometry as follows:
¹H NMR (400 MHz, DMSO-d₆); peaks at b 9.05 (s), 7.54 (s), 7.06 (m), 6.84 (m), 6.60 (m), 5.56, 4.85, 3.77 (m), 1.51 (m), 1.23 (s), and 0.84 (t) ppm, and
MS: 513 (M+H—Na), 511 (M-H—Na).

### Example 15

### Chemical stability of the fosfluconazole derivative sodium 2-(2,4-difluorophenyl)-1,3-di(1H-1,2,4-triazol-1-yl)propan-2-yl nonyl phosphatein pharmaceutical solvents

Table 10 provides the chemical stability data in some pharmaceutical solvents, expressed as the weight percentage (% w/w) of the final fosfluconazole derivative produced and characterized in example 14 that remained in solution after a certain period of time. The test was carried out twice, and the remaining weight % was determined by Liquid Chromatography-Mass Spectrometry (LCMS).

TABLE 10

| Pharmaceutical solvents | 4 hours | 1 day | 2 days | 7 days | 14 days |
|---|---|---|---|---|---|
| H₂O (pH = 2) | 99.0 | 99.1 | 99.2 | 99.2 | 99.6 |
| H₂O (pH = 7) | 99.1 | 99.3 | 99.2 | 99.2 | 99.4 |
| H₂O (pH = 10) | 99.3 | 99.3 | 99.4 | 99.3 | 99.9 |
| H₂O/Kleptose HPB (60/40) | 99.5 | 99.3 | 99.2 | 99.3 | 100.0 |
| PEG 400 | 99.3 | 99.3 | 99.4 | 99.4 | 100.0 |

Table 10 shows that at most 0.6% of the fluconazole derivative did not remain in the relevant aqueous or hydroxypropyl-β-cyclodextrin or polyethylene glycol solution after 14 days.

1. A compound of formula (I) 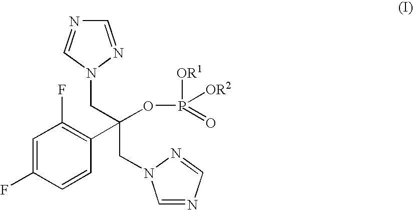
and the salts, N-oxides, quaternary amines, and stereoisomers thereof, wherein
R¹ is hydrogen or C₁₋₆alkyl;
R² is a steranyl or a group selected from 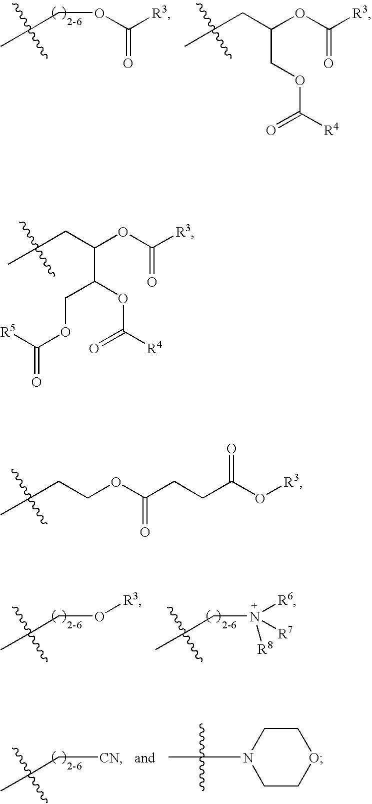
the symbol 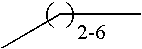
represent a C₂₋₆alkanediyl;
R³, R⁴, and R⁵ are each independently, C₆₋₁₈alkyl or steranyl; and
R⁶, R⁷, and R⁸ are each, independently, C₁₋₆alkyl.

2. The compound according to claim 1, wherein R² is a steranyl or a group selected from

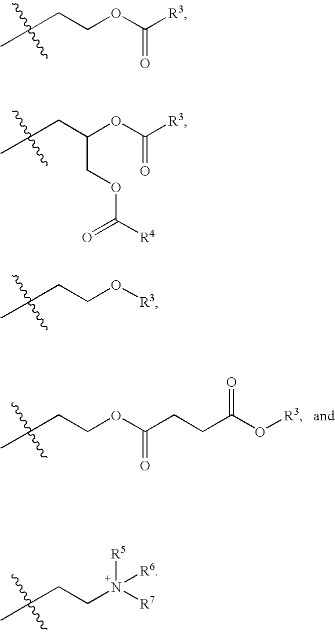

3. The compound according to claim 1, wherein
R² is a steranyl or a group 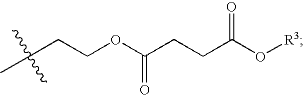
in which group, R³ is steranyl.

4. The compound according to any one of claims 1-3, wherein the steranyl is cholesteranyl.

5. The compound according to any one of claims 1-4, wherein R¹ is ethyl.

6. A salt of the compound according to any one of claims 1-4, wherein R¹ is hydrogen and the salt is a monosodium salt.

7. A pharmaceutical composition comprising a pharmaceutically acceptable carrier, and as active ingredient an effective amount of the compound according to any one of claims 1-6 or a pharmaceutically acceptable salt thereof.

8. The pharmaceutical composition according to claim 7, wherein said pharmaceutical composition is administered intravenously, intramuscularly, subcutaneously, intraperitoneally, intra-articularly, intralesionally, intraventricularly, by spinal injection, by intraosseous infusion, or transdermally.

9. A compound according to any one of claims 1-6 or a pharmaceutically acceptable salt thereof, for use as a medicament.

10. Use of a compound according to any one of claims 1-6 or a pharmaceutically acceptable salt thereof, for the manufacture of a medicament for the prevention or treatment of fungal infections.

11. A compound according to any one of claims 1-6 or a pharmaceutically acceptable salt thereof, for use in the prevention or treatment of fungal infections

12. A method of prevention or treatment of fungal infections, which comprises administering an effective amount of a compound according to any one of claims 1-6 or a pharmaceutically acceptable salt thereof, to a patient in need of such prevention or treatment.

13. A process for the preparation of a compound of formula (I) according to any one of claims 1-6 or a pharmaceutically acceptable salt thereof, which comprises
a1) reacting fluconazole with a phosphoramidite of formula (II) in a suitable reaction medium thereby obtaining a phosphite of formula (III), and further reacting said phosphite of formula (III) with an oxidant; or 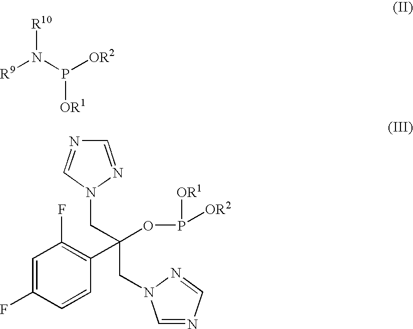
a2) reacting fluconazole with a phosphorochloridate of formula (IV) in a suitable anhydrous reaction medium; or 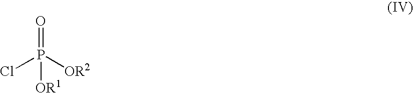
a3) reacting fluconazole with PCl₃ in the presence of a base thereby obtaining an intermediate compound of formula (V),
R—O—PCl₂ (V)
and reacting compound of formula (V) with a compound of formula R¹—OH and a compound of formula R²—OH; and
b) optionally converting the resulting compound into a pharmaceutically acceptable salt or vice versa;
wherein
R¹ and R² are each as defined in claim 1;
R⁹ and R¹⁰ are each, independently, C₁₋₆alkyl optionally substituted with C₃₋₇cycloalkyl or phenyl, optionally substituted phenyl, or C₃₋₇cycloalkyl; or R⁹ and R¹⁰ together with the nitrogen atom to which they are attached form an optionally substituted 5- or 6-membered saturated heterocyclic ring, wherein the substituents may be selected from C₁₋₄alkyl and phenyl; and
R is

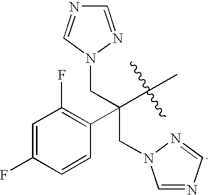

14. A phosphoramidite of formula (II), a phosphite of formula (III), or a phosphorochloridate of formula (IV), as defined in claim 13.

15. Use of the chemical group of formula (VI) as a promoiety 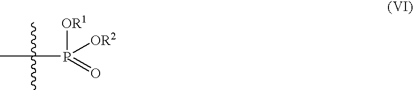
wherein R¹ and R² are each as defined in claim 1; and
the wavy line (depicted by [symbol] )indicates the bond to the oxygen atom of a drug.

16. A method of improving the lipophilicity of fluconazole, which comprises converting said fluconazole into a compound according to any one of claims 1-6 or a pharmaceutically acceptable salt thereof.

17. A method of extending the release of fluconazole, which comprises converting fluconazole into a compound according to any one of claims 1-6 or a pharmaceutically acceptable salt thereof.
